(19) United States
(12) Patent Application Publication
(10) Pub. No.: US 2018/0289843 A1
(43) Pub. Date: Oct. 11, 2018

(54) COATING FOR METAL NANOPARTICLES

(71) Applicants: AGENCY FOR SCIENCE, TECHNOLOGY AND RESEARCH, Singapore (SG); UNIVERSITY OF LIVERPOOL, Liverpool (GB)

(72) Inventors: David PARAMELLE, Singapore (SG); David FERNIG, Liverpool (GB); Katie WILSON, Liverpool (GB)

(21) Appl. No.: 15/764,317

(22) PCT Filed: Sep. 28, 2016
(86) PCT No.: PCT/SG2016/050480

§ 371 Date: Mar. 28, 2018

(30) Foreign Application Priority Data
Sep. 28, 2015 (SG) 10201508038W

(51) Int. Cl. A61K 49/18; C07K 7/06

(52) U.S. Cl. CPC A61K 49/1866; C07K 7/06; A61K 49/1842

(57) ABSTRACT

The invention relates to a ligand compound having a structure A-B-C, wherein (a) A represents a mono- or polyphosphorylated amino acid linked to part B by its amino group to form an amide bond; B represents (i) a carboxylic acid, and (ii) an amino acid or peptidyl group of 2-10 amino acids, an alkyl or alkenyl group comprising 1-26 carbon atoms, a polyethylene glycol group comprising 1-26 carbon atoms or a combination thereof covalently linked to the carboxylic acid; and C represents a hydrophilic group covalently linked to the group of B (ii) or (b) A represents a mono-or polyphosphorylated amino acid linked to B by its carboxylic acid to form an amide bond; B represents an amino acid or peptidyl group of 2-10 amino acids, an amino substituted alkyl or alkenyl group comprising 1-26 carbon atoms, an amino substituted polyethylene glycol group comprising 1-26 carbon atoms or a combination thereof covalently linked to A by their amino group; C represents a hydrophilic group covalently linked to the group of B. The invention further relates to a coated metal nanoparticle such as super paramagnetic iron oxide nanoparticle (SPIONs) coated with a plurality of the aforementioned ligands and a method of producing thereof.

### CROSS-REFERENCE TO RELATED APPLICATION

This application claims the benefit of priority of Singapore Patent Application No. 10201508038W, filed 28 Sep. 2015, the contents of which being hereby incorporated by reference in its entirety for all purposes.

### FIELD OF THE INVENTION

The present invention lies in the field of biochemistry and relates to a ligand compound having the structure A-B-C, wherein (a) A represents a mono- or polyphosphorylated amino acid linked to part B by its amino group to form an amide bond; B represents (i) a carboxylic acid linked to part A by its acidic group to form the amide bond, and (ii) an amino acid or peptidyl group of 2 to 10 amino acids, an alkyl or alkenyl group comprising 1 to 26 carbon atoms, a polyethylene glycol group comprising 1 to 26 carbon atoms or a combination thereof covalently linked to the carboxylic acid; and C represents a hydrophilic group covalently linked to the group of B (ii); or (b) A represents a mono- or polyphosphorylated amino acid linked to part B by its carboxylic acid to form an amide bond; B represents an amino acid or peptidyl group of 2 to 10 amino acids, an amino substituted alkyl or alkenyl group comprising 1 to 26 carbon atoms, an amino substituted polyethylene glycol group comprising 1 to 26 carbon atoms or a combination thereof covalently linked to A by their amino group; C represents a hydrophilic group covalently linked to the group of B. The present invention further relates to a coated metal nanoparticle comprising the ligand compound. In addition, the present invention also relates to the use and methods of production of the ligand compound and the coated metal nanoparticle of the invention.

### BACKGROUND OF THE INVENTION

In order to use nanoparticles in biological applications, they need to be coated by a ligand shell (called biofunctionalisation) to provide stability in a physiological environment, including preventing non-specific binding, and to target the nanoparticle to areas of interest in a sample. One approach to synthesising ligand shells is to self-assemble a monolayer of small ligands on the surface of the nanoparticle. The ligand can be considered to consist of a “head”, “stem” and “foot”. The “foot” serves to anchor the ligand to the surface of the nanoparticle and, with the “stern”, drive self-assembly of the shell and seal off the core material from the environment. The environment is only exposed to the “head” at the distal end of the “stem”. While the “stem” and “head” groups could be easily transposed to many different kinds of nanoparticles, the “foot” must be adapted according to the surface properties of the nanoparticle. This approach has hitherto been successful with noble metal nanoparticles.

One type of nanoparticles that are important in biological applications are superparamagnetic iron oxide nanoparticles (SPIONs). SPIONs, because of their magnetic properties and biocompatibility in vivo (multiple iron oxide nanoparticles based products have been FDA approved, e.g., Resovist), are particularly attractive materials for enhancing magnetic resonance imaging contrast in a variety of in vivo situations. It is noted that the thiol “foot” of EG alkanethiol would not bind well to iron oxide and hence not ideal for the passivation of the surface of iron oxide nanoparticles.

Qu et al. (Qu, H. et al., Langmuir 2014, 30, 10918-10925) discloses large polyethylene glycol ligands (Mn 5000) to prepare coated iron oxide nanoparticles. However, for the use of some biological applications smaller coated iron oxide nanoparticles are desirable. US 20090208420 A1 discloses binding peptide of 5-100 (amino acid) units. Barch et al. (Barch, M. et al., J. Am. Chem. Soc. 2014, 136, 12516-12519) discloses peptides binding iron oxide nanoparticles surface to prepare water soluble iron oxide nanoparticles. Nonetheless, US 20090208420 A1 and Barch et al. do not suggest highly specific “foot” moieties for the use of coating iron oxide nanoparticles.

Hence, there is need in the art for methods and devices to improve the coating of metal (e.g. iron) oxide nanoparticles.

### SUMMARY OF THE INVENTION

It is an object of the present invention to meet the above need by providing a ligand compound according to the invention. Surprisingly, the present inventors have found that ligand compounds of the invention has good colloidal property in water, resistance to non-specific binding to charged surfaces or biomolecules, and colloidal stability in electrolytes via efficient steric repulsion. Said ligand compounds are self-assembling to provide a coating on an iron oxide nanoparticle. Further, they are biocompatible, up-scalable for in vivo applications and can provide biofunctionalization for targeting applications. In addition, the ligand compounds of the present invention are the first peptide coating based on phosphorylated amino acid for iron oxide nanoparticles. This will provide a thin protecting layer on nanoparticles surface. Moreover, the present ligand compounds enable the production of the first peptide coated iron oxide nanoparticles having high stability over a long period in harsh biological environments. This is the key for medical applications such as MRI.

In a first aspect, the present invention is thus directed to a ligand compound having the structure A-B-C, wherein (a) A represents a mono- or polyphosphorylated amino acid linked to part B by its amino group to form an amide bond; B represents (i) a carboxylic acid linked to part A by its acidic group to form the amide bond, and (ii) an amino acid or peptidyl group of 2 to 10 amino acids, an alkyl or alkenyl group comprising 1 to 26 carbon atoms, a polyethylene glycol group comprising 1 to 26 carbon atoms or a combination thereof covalently linked to the carboxylic acid; and C represents a hydrophilic group covalently linked to the group of B (ii); or (b) A represents a mono- or polyphosphorylated amino acid linked to part B by its carboxylic acid to form an amide bond; B represents an amino acid or peptidyl group of 2 to 10 amino acids, an amino substituted alkyl or alkenyl group comprising 1 to 26 carbon atoms, an amino substituted polyethylene glycol group comprising 1 to 26 carbon atoms or a combination thereof covalently linked to A by their amino group; C represents a hydrophilic group covalently linked to the group of B.

In various embodiments of the invention, (a) the phosphorylated amino acid is phosphoserine, phosphothreonine or phosphotyrosine and/or (b) the phosphorylated amino acid of the ligand compound according to alternative (b) is an amino acid comprising attached to its N-terminus the moiety PO₃H₂—O—CH₂—CO—. The scope of the present invention also encompasses various embodiments wherein the carboxylic acid is an amino acid.

In still further various embodiments of the invention, the hydrophilic group is a group comprising a carboxyl group, a hydroxyl group or an amine group.

In various embodiments, the hydrophilic group is an amino acid derivative selected from the group consisting of aspartyl, glutaminyl, arginyl, histidyl and lysyl.

In further various embodiments of the invention, the ligand compound is functionalized by the attachment of an additional group. In more preferred embodiments, the additional group is selected from the group consisting of a dye, a radionuclide, a pharmaceutical agent, a biotherapeutic agent, a chemotherapeutic agent, a radiotherapeutic agent, and combinations thereof.

Also encompassed are embodiments, wherein (a) the compound having the structure of formula (I)

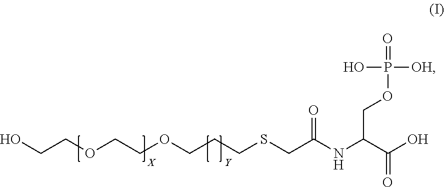

wherein X and Y are independently from each other an integer ranging from 1-10; (b) the ligand compound is selected from the group consisting of H-Ser-(PO₃H₂)—NH-PEG₄-ol, PO₃H₂—O—CH₂—CO-Gly-NH-PEG₄-OH, PO₃H₂—O—CH₂—CO-Ser(PO₃H₂)—NH-PEG₄-OH, PO₃H₂—O—CH₂—CO-Ser(PO₃H₂)—NH-PEG₄-ol, H-Ser-(PO₃H₂)-Ser-Ser-Ser-Ser-ol, H-Ser-(PO₃H₂)-Val-Val-Val-Thr-ol and PO₃H₂—O—CH₂—CO-Ser(PO₃H₂)-Val-Val-Val-Thr-ol.

In more preferred embodiments, X is 3 and Y is 9.

In a further aspect, the present invention relates to a coated metal nanoparticle comprising a core metal nanoparticle that is coated with a plurality of ligand compounds of the invention.

In various embodiments of the invention, the plurality of ligand compounds of the invention comprises a mixture of at least two structurally different ligand compounds.

The scope of the present invention also encompasses various embodiments wherein the core metal nanoparticle is a metal oxide nanoparticle, preferably iron oxide nanoparticle and more preferably a superparamagnetic iron oxide nanoparticle (SPION).

In a still further aspect of the invention, the scope encompasses the use of a ligand compound of the invention for coating a metal nanoparticle.

In a fourth aspect, the present invention relates to a method of producing a coated metal nanoparticle of the invention comprising: (a) providing a core metal nanoparticle and a plurality of ligand compounds of the invention; and (b) combining the core metal nanoparticle and the plurality of ligand compounds under conditions that allow the formation of the coated metal nanoparticle of the invention.

In various embodiments, the above method comprises prior to step (a) encapsulation of the metal nanoparticle with an intermediate hydrophilic ligand, preferably tetramethylammonium hydroxide (TMAOH).

In a further aspect, the invention relates to coated metal nanoparticle of the invention for use as a medicament.

In various embodiments of the coated metal nanoparticle for use, the ligand compound is functionalized by the attachment of an additional group.

The scope of the present invention also encompasses various embodiments wherein the additional group is selected from the group consisting of a dye, a radionuclide, a pharmaceutical agent, a biotherapeutic agent, a chemotherapeutic agent, a radiotherapeutic agent, and combinations thereof.

In a sixth aspect, the invention relates to a method of producing the ligand compound of the invention comprising:

a) reacting a compound of formula (II)

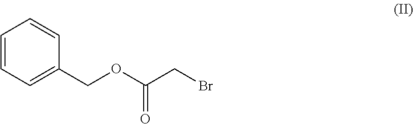 [SMILES: O=C(CBr)OCc1ccccc1]

with NaI to form a compound of formula (III)

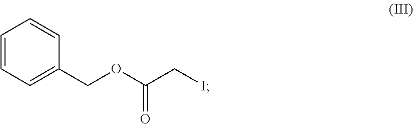 [SMILES: O=C(CI)OCc1ccccc1]

b) reacting the compound of formula (III) with a compound of formula (IV)

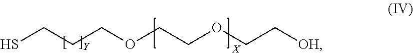

wherein X and Y are independently from each other an integer ranging from 1-10, to form a compound of formula (V)

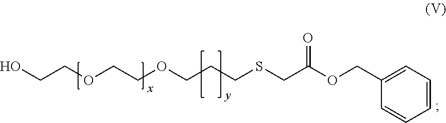

c) reacting the compound of formula (V) with Boc₂O to form a compound of formula (VI)

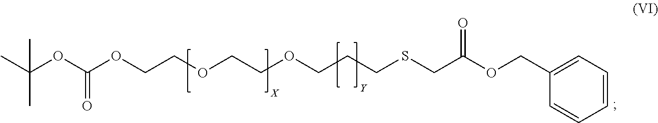

d) reacting the compound of formula (VI) with H₂ to form the a compound of formula (VII)

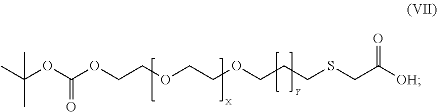

e) reacting the compound of formula (VII) with a compound of formula (VIII)

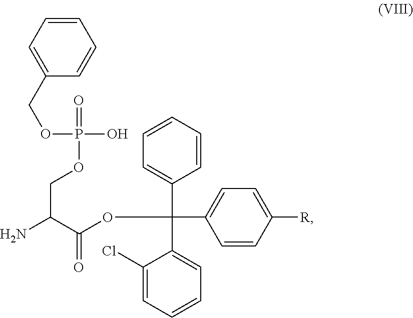

wherein R represents a resin,
to form a compound of formula (IX)

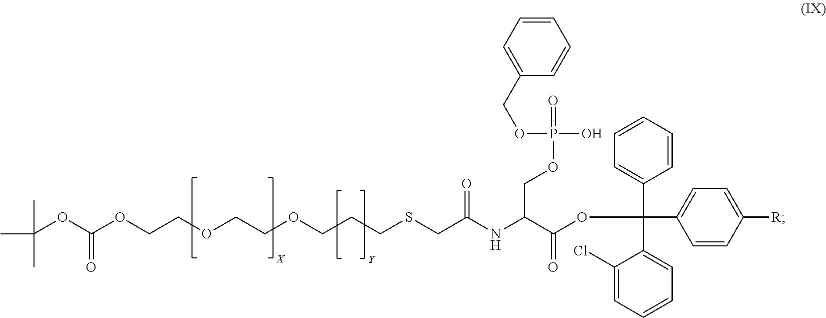

f) reacting the compound of formula (IX) with dichloromethane (DCM): trifluoroacetic acid (TFA) to form a compound of formula (X)

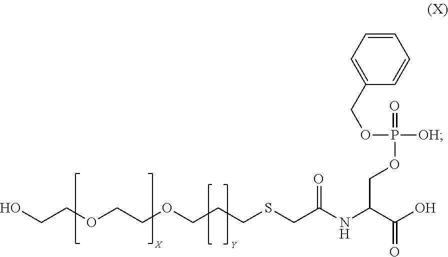

and
g) reacting the compound of formula (X) with H₂ to form the ligand compound of the invention.

### BRIEF DESCRIPTION OF THE DRAWINGS

The invention will be better understood with reference to the detailed description when considered in conjunction with the non-limiting examples and the accompanying drawings.

FIG. 1 shows a scheme of an EG alkane phosphoserine ligand for coating of SPIONs.

FIG. 2 shows a scheme for a strategy for the preparation of a ligands library based on a peptide sequence.

FIG. 3 shows the exchange of oleic acid ligand on SPIONs for EG alkanethiol phosphoserine. SPIONs coated in oleic acid and soluble in toluene (A) underwent ligand-exchange to produce EG alkanethiol phosphoserine capped SPIONs that were soluble in aqueous solutions (B).

FIG. 4 shows Sephadex G25 size-exclusion chromatography of water soluble EG alkanethiol phosphoserine capped SPIONs. SPIONs were subjected to Sephadex G25 chromatography after the first incubation with EG alkanethiol phosphoserine ligand. Images of (A) the SPIONs on the column and (B) the SPIONs that eluted from the column in the excluded volume, Vo.

FIG. 5 shows chromatography of EG alkanethiol phosphoserine capped SPIONs on ion-exchange chromatography resins. After the final incubation with EG alkanethiol phosphoserine ligand, SPIONs were transferred to water and concentrated and then subjected to DEAE anion-exchange and CM cation-exchange chromatography. Images were acquired of (A) the SPIONs on the DEAE and CM resins, (B) the SPIONs washed from the resins with the water and (C) of the DEAE and CM resins after the water washes.

FIG. 6 shows the dissolution of SPIONs in citrate at different pHs. SPIONs were incubated with sodium citrate at pH 7.14, pH 5.5 and pH 4.5 for the numbers of days indicated before adding Ferrozine reagent. The percentage dissolution of the SPIONs was then determined by measuring the amount of Fe³⁺ ions in solution using the UV-Visible absorbance of Ferrozine chelated to Fe³⁺ at 590 nm.

FIG. 7 shows the ligand exchange procedures of oleic acid coated iron oxide nanoparticles with hydrophilic peptide ligands. A/ Direct transfer by mixing of oleic acid coated nanoparticles in organic solvent with aqueous solution of peptides (1,2) and removal of organic phase. B/ Water transfer of oleic acid coated nanoparticles from organic solvent with an intermediate hydrophilic ligand (4), removal of organic phase (5) and ligand exchange of intermediate ligand coating with peptide ligand (6).

FIG. 8 shows oleic acid coated iron oxide nanoparticles in CHCl₃ (A) transferred into a 2 mM aqueous solution of TMAOH.

FIG. 9 shows the stability of TMAOH coated iron oxide nanoparticles in (A) 2 mM TMAOH aqueous after two days and (B) PBS buffer after one hour.

FIG. 10 shows the non-specific binding evaluation of TMAOH coated iron oxide nanoparticles on (A) G25, (B) DEAE and (C) CM resins columns.

FIG. 11 shows electrolyte-induced aggregation stability test of single peptide ligand coated iron oxide nanoparticles using (A) peptide S7, (B) peptide S8, (C) peptide S13 and (D) peptide S14. A normalized aggregation parameter equal to one indicate high stability of the nanoparticles.

FIG. 12 shows electrolyte-induced aggregation stability test of single peptide ligand coated iron oxide nanoparticles prepared with peptide S14, mixed in (A) 1 M NaCl at room temperature, (B) PBS buffer at room temperature and (C) PBS buffer at 37° C. for two days. Here are presented the UV-visible spectra.

FIG. 13 shows electrolyte-induced aggregation stability test of mixed peptide ligand coated iron oxide nanoparticles using peptide S14 with (A) ligand L1, (B) ligand L2 and (C) ligand L3 with a molar ratio peptide:ligand of 70:30. A normalized aggregation parameter equal to one indicates high stability of the nanoparticles.

FIG. 14 shows electrolyte-induced aggregation stability test of mixed peptide ligand coated iron oxide nanoparticles prepared with peptide S14 and ligand L1, mixed in (A) 1 M NaCl at room temperature, (B) PBS buffer at room temperature and (C) PBS buffer at 37° C. for two days. Here are presented the UV-visible spectra.

FIG. 15 shows a MRI image of L1(30%)+S9(70%) mixed peptide coated nanoparticles and its corresponding 1/T2 vs Fe²⁺ plot.

FIG. 16 shows a MRI image of peptide S7 coated nanoparticles and its corresponding 1/T2 vs Fe²⁺ plot.

FIG. 17 shows the intensity vs Echo Time curve of Raym 8-5 (0.36 mM) showing bad fitting.

FIG. 18 shows a MTT in vitro cytotoxicity assay of peptide coated iron oxide nanoparticles with BT474 breast cancer cells.

FIG. 19 shows the non-specific binding assay of peptide coated iron oxide nanoparticles with BT474 breast cancer cells staining with Prussian blue.

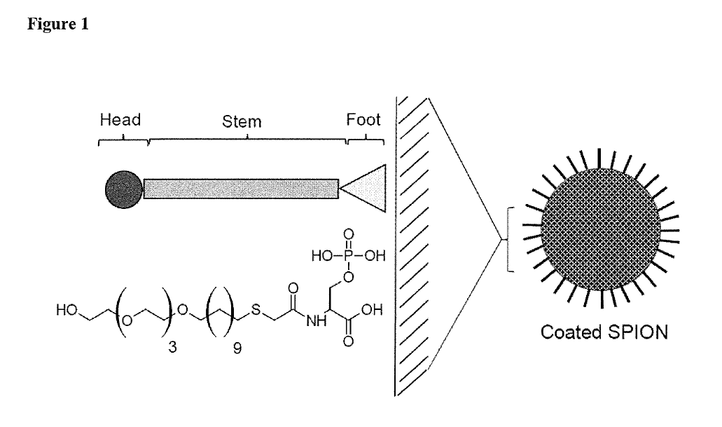

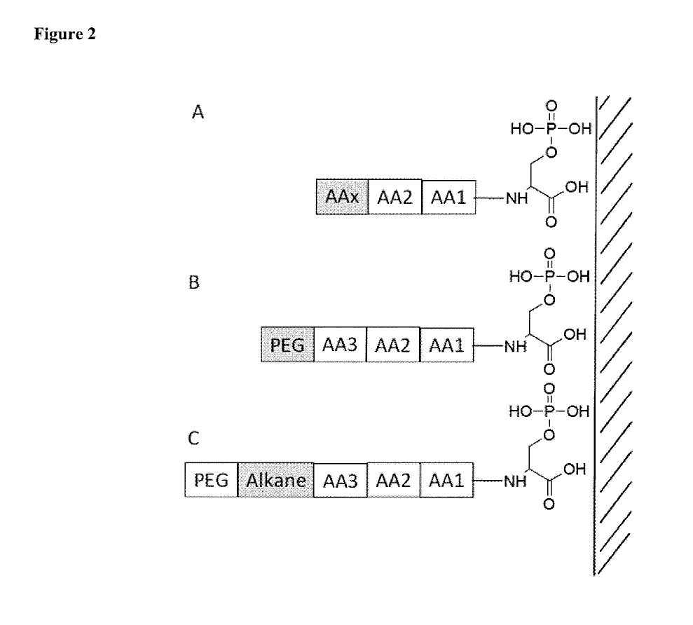

### DETAILED DESCRIPTION OF THE INVENTION

The present inventors surprisingly found that ligand compounds as described herein and comprising a phosphorylated amino acid are able to bind to core iron oxide nanoparticles to form a coating around said nanoparticle. These coatings are established upon self-assembly by bringing the ligand compounds and the nanoparticle in contact with each other. The resulting coated nanoparticles are biocompatible and can be functionalized by linking them to chemical moieties or groups that provide specific properties. Due to the small length of the ligand compound (for example 1-10 amino acids), the coated nanoparticles have only a thin protecting layer on their surface. Said coated nanoparticles are highly stabile over a long period in harsh biological environments, a criterion important for their application in medical use.

Therefore, in a first aspect, the present invention is thus directed to a ligand compound having the structure A-B-C, wherein (a) A represents a mono- or polyphosphorylated amino acid linked to part B by its amino group to form an amide bond; B represents (i) a carboxylic acid linked to part A by its acidic group to form the amide bond, and (ii) an amino acid or peptidyl group of 2 to 10 amino acids, an alkyl or alkenyl group comprising 1 to 26 carbon atoms, a polyethylene glycol group comprising 1 to 26 carbon atoms or a combination thereof covalently linked to the carboxylic acid; and C represents a hydrophilic group covalently linked to the group of B (ii); or (b) A represents a mono- or polyphosphorylated amino acid linked to part B by its carboxylic acid to form an amide bond; B represents an amino acid or peptidyl group of 2 to 10 amino acids, an amino substituted alkyl or alkenyl group comprising 1 to 26 carbon atoms, an amino substituted polyethylene glycol group comprising 1 to 26 carbon atoms or a combination thereof covalently linked to A by their amino group; C represents a hydrophilic group covalently linked to the group of B.

The terms “ligand” or “ligand compound” as interchangeably used herein, refers to a molecule or more generally to a compound which is capable of binding to the target molecule. The ligand can bind to the target molecule with any affinity i.e. with high or low affinity. Generally, a ligand which binds to the target molecule with high affinity may result in a more thermally stable target molecule compared to a ligand which binds to the target molecules with a lower affinity. In preferred embodiments, the interaction between the ligand and the target molecule is non-covalently. Typically, a ligand capable of binding to a target molecule may result in the thermal stabilization of that target molecule by at least 0.25 or 0.5° C. and preferably at least 1, 1.5 or 2° C. In the present invention, the target molecule is an (core) iron oxide nanoparticle. The ligand compound is the compound as defined above consisting of or comprising parts A, B and C. In preferred embodiments of the invention, the ligand compound does not contain more than 50, not more than 45, not more than 40, not more than 35, not more than 30, not more than 25, not more than 20, not more than 19, not more than 18, not more than 17, not more than 16, not more than 15, not more than 14, not more than 13, not more than 12, not more than 11, not more than 10, not more than 9, not more than 8, not more than 7, not more than 6 or not more than 5 carbon atoms in total (referring to the sum of parts A, B and C). In other preferred embodiments, part A of the ligand compound comprises or consists of a phosphorylated amino acid, such as phosphoserine, phosphothreonine or phosphotyrosine. However, the phosphorylated amino acid may also be any phosphorylated non-proteinogenic amino acid. Part B of the ligand compound comprises or consists of (i) a carboxylic acid and (ii) an amino acid or peptidyl group of 2 to 10 amino acids (preferably 3-9, 4-8 or 5-7), an alkyl or alkenyl group comprising 1 to 26 carbon atoms (preferably 2-20, 3-18, 4-15, 6-10), a polyethylene glycol group comprising 1 to 26 carbon atoms (preferably 2-20, 3-18, 4-15, 6-10)or a combination thereof. Both parts are covalently linked by a linkage, such as but not limited to C—C, ester, ether, thioester, thioether or amide bondage. The carboxylic acid may comprise or consist of not more than 14, not more than 12, not more than 10, not more than 9, not more than 8, not more than 7, not more than 6, not more than 5 or not more than 4 carbon atoms. In other preferred embodiments, part C represents a hydrophilic group that comprises or consists of not more than 14, not more than 12, not more than 10, not more than 9, not more than 8, not more than 7, not more than 6, not more than 5 or not more than 4 carbon atoms.

As used herein, the terms “phosphorylation” or “phosphorylated” refer to the process of covalently adding one or more phosphate groups to a molecule (e.g., to an amino acid).

The term “amino acid”, as used herein, means the stereoisomers forms, e.g. D and L forms, of proteinogenic and non-proteinogenic amino acids. These amino acids comprise, but are not limited to the following compounds: alanine, β-alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine, κ-aminobutyrate, Ne-acetyllysine, Nd-acetylornithine, N_{κ}-acetyldiaminobutyrate and No-acetyldiaminobutyrate. L-amino acids are preferred.

Basic amino acids are polar and positively charged at pH values below their pKa's, and are very hydrophilic; histidine, lysine and arginine are basic amino acids. Acidic amino acids are negatively charged, polar and hydrophilic and include aspartic acid and glutamic acid.

The term “peptide” encompasses a sequence of two or more amino acids wherein the amino acids are naturally occurring or synthetic (non-naturally occurring) amino acids. The term “peptide” typically refers to short polypeptides.

As used herein, the terms “bound” and “linked” refer to binding or attachment that may be covalent, e.g., by chemically coupling, or non-covalent, e.g., ionic interactions, hydrophobic interactions, hydrogen bonds, etc. Covalent bonds can be, for example, ester, ether, phosphoester, thioester, thioether, urethane, amide, amine, peptide, imide, hydrazone, hydrazide, carbon-sulfur bonds, carbon-phosphorus bonds, and the like. The above terms are broader than and include terms such as “coupled”, “conjugated” and “attached”.

The term “carboxylic acid”, as used herein, means a carboxylic acid of formula R—C(O)OH, wherein R is a C1-C14 hydrocarbon group. In one embodiment, R is a C1-C8 hydrocarbon group. In one embodiment, the C1-C14 hydrocarbon group is substituted, such as an —OH group or a —NH₂ group.

The phrase a “C1-C14 hydrocarbon group”, as used herein, means a straight or branched, saturated or unsaturated, cyclic or non-cyclic, carbocyclic group having from 1 to 14 carbon atoms. Similarly, the phrases a “C1-C8 hydrocarbon group” means a straight or branched, saturated or unsaturated, cyclic or non-cyclic, carbocyclic group having from 1 to 8 carbon atoms.

The term “alkyl”, as used herein refers to a linear, branched, or cyclic saturated hydrocarbon group typically although not necessarily containing 1 to about 26 carbon atoms, preferably 1 to about 12 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although not necessarily, alkyl groups herein contain 1 to about 12 carbon atoms. The term “lower alkyl” intends an alkyl group of 1 to 6 carbon atoms, and the specific term “cycloalkyl” intends a cyclic alkyl group, typically having 4 to 8, preferably 5 to 7, carbon atoms. If not otherwise indicated, the terms “alkyl” and “lower alkyl” include linear, branched, cyclic, unsubstituted, or substituted alkyl and lower alkyl, respectively.

The term “alkenyl”, as used herein, is a hydrocarbon group of from 2 to 26 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “polyethylene glycol group”, as used herein, refers to a moiety consisting of one or more polyethylene glycol units, such as —(OCH₂CH₂)ₓ₁O—, wherein X1 represent the number of polyethylene glycol units (not more than 13) and — represents the binding to the other groups of the ligand compound, e.g. the hydrophilic group and the carboxylic acid.

The terms “covalent” or “covalently”, as used herein, refer to the nature of a chemical bonding interaction between atoms. A covalent bond is a chemical bonding that involves the sharing of electron pairs between atoms. The stable balance of attractive and repulsive forces between atoms when they share electrons is referred to as covalent bonding. The sharing of electrons allows each atom to attain the equivalent of a full outer shell, corresponding to a stable electronic configuration. Covalent bonding includes various kinds of interactions, e.g., σ-bonding, π-bonding, metal-to-metal bonding, agnostic interactions, and three-center two-electron bonds.

The term “hydrophilic” as it relates to part C of the ligand compound of the invention does not essentially differ from the common meaning of this term in the art, and denotes organic moieties which contain ionizable, polar, or polarizable atoms, or which otherwise may be solvated by water molecules. Thus a hydrophilic group, as used herein, refers to an aliphatic, alicyclic, heteroaliphatic, heteroalicyclic, aryl or heteroaryl moiety, which falls within the definition of the term hydrophilic, as defined above. Examples of particular hydrophilic organic moieties which are suitable include, without limitation, aliphatic or heteroaliphatic groups comprising a chain of atoms in a range of between about one and twelve atoms, hydroxyl, hydroxyalkyl, amine, carboxyl, amide, carboxylic ester, thioester, aldehyde, nitryl, isonitryl, nitroso, hydroxylamine, mercaptoalkyl, heterocycle, carbamates, carboxylic acids and their salts, sulfonic acids and their salts, sulfonic acid esters, phosphoric acids and their salts, phosphate esters, polyglycol ethers, polyamines, polycarboxylates, polyesters and polythioesters. In various embodiments of the invention, the hydrophilic group is a group comprising or consisting of a carboxyl group, a hydroxyl group or an amine group.

In various embodiments of the invention, (a) the phosphorylated amino acid is phosphoserine, phosphothreonine or phosphotyrosine and/or (b) the phosphorylated amino acid of the ligand compound according to alternative (b) is an amino acid comprising attached to its N-terminus the moiety PO₃H₂—O—CH₂—CO—.

In various embodiments of the invention, the phosphorylated amino acid is phosphoserine, phosphothreonine or phosphotyrosine. The term “phosphoserine” refers to a compound having the following formula:

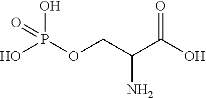 [SMILES: NC(COP(=O)(O)O)C(=O)O]

The term “phosphothreonine” refers to a compound having the following formula:

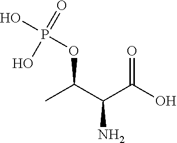

The term “phosphotyrosine” refers to a compound having the following formula:

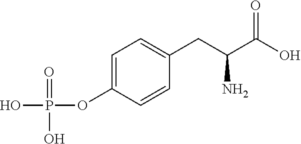 [SMILES: N[C@@H](Cc1ccc(OP(=O)(O)O)cc1)C(=O)O]

For each of the phosphorylated amino acids, including phosphoserine, phosphothreonine and phosphotyrosine, the NH₂-group is linked to the acidic group of the carboxylic acid to form an amide bond.

The term “monophosphorylated”, as used herein in the context of amino acids, means that the amino acid contains one phosphorylation group. The term “polyphosphorylated”, as used herein in the context of amino acids, means that the amino acid contains at least two phosphorylation groups. In preferred embodiments, the amino acid contains two phosphorylation groups, thus said amino acid is di-phosphorylated. The phosphorylation groups may be attached to the carboxylic group, the amino group or to other chemical groups, such as alcoholic groups. In the case of a polyphosphorylation, the amino acid may comprise a mixture of the above described attachments.

The scope of the present invention also encompasses various embodiments wherein the carboxylic acid is an amino acid.

In various embodiments, the hydrophilic group is an amino acid derivative selected from the group consisting of aspartyl, glutaminyl, arginyl, histidyl and lysyl.

In further various embodiments of the invention, the ligand compound is functionalized by the attachment of an additional group. The term “functionalized” or “functionalized group”, as used herein, means an atom or group of atoms, acting as a unit, that replaces a hydrogen atom in the ligand compound, and whose presence imparts characteristic properties to the molecule. In more preferred embodiments, the additional group is selected from the group consisting of a dye, a radionuclide, a pharmaceutical agent, a biotherapeutic agent, a chemotherapeutic agent, a radiotherapeutic agent, and combinations thereof.

The dye can be either a “small molecule” dye/fluors, or a proteinaceous dye/fluors (e.g. green fluorescent proteins and all variants thereof). Suitable dyes include, but are not limited to, 1,1′-diethyl-2,2′-cyanine iodide, 1,2-diphenylacetylene, 1,4-diphenylbutadiene, 1,6-Diphenylhexatriene, 2-Methylbenzoxazole, 2,5-Diphenyloxazole (PPO), 4-(dicyanomethylene)-2-methyl-6-(p-dimethylaminostyryl)-4H-pyran (DCM), 4-Dimethylamino-4′-nitrostilbene, 4′,6-Diamidino-2-phenylindole (DAPI), 5-ROX, 7-AAD, 7-Benzylamino-4-nitrobenz-2-oxa-1,3-diazole, 7-Methoxycoumarin-4-acetic acid, 9,10-Bis(phenylethynyl)anthracene, 9,10-Diphenylanthracene, Acridine Orange, Acridine yellow, Adenine, Allophycocyanin (APC), AMCA, AmCyan, Anthracene, Anthraquinone, APC, Auramine 0, Azobenzene, Benzene, Benzoquinone, Beta-carotene, Bilirubin, Biphenyl, BO-PRO-1, BOBO-1, BODIPY FL, Calcium Green-1, Cascade Blue™, Cascade Yellow™, Chlorophyll a, Chlorophyll b, Chromomycin, Coumarin, Coumarin 1, Coumarin 30, Coumarin 314, Coumarin 343, Coumarin 6, Cresyl violet perchlorate, Cryptocyanine, Crystal violet, Cy2, Cy3, Cy3.5, Cy5, Cy5.5, Cy7, Cytosine, DA, Dansyl glycine, DAPI, DiI, DiO, DiOCn, Diprotonated-tetraphenylporphyrin, DsRed, EDANS, Eosin, Erythrosin, Ethidium Monoazide, Ethyl p-dimethylaminobenzoate, FAM, Ferrocene, FI, Fluo-3, Fluo-4, Fluorescein, Fluorescein isothiocyanate (FITC), Fura-2, Guanine, HcRed, Hematin, Histidine, Hoechst, Hoechst 33258, Hoechst 33342, IAEDANS, Indo-1, Indocarbocyanine (C3)dye, Indodicarbocyanine (C5)dye, Indotricarbocyanine (C7)dye, LC Red 640, LC Red 705, Lucifer yellow, LysoSensor Yellow/Blue, Magnesium octaethylporphyrin, Magnesium octaethylporphyrin (MgOEP), Magnesium phthalocyanine (MgPc), Magnesium tetramesitylporphyrin (MgTMP), Magnesium tetraphenylporphyrin (MgTPP), Malachite green, Marina Blue®, Merocyanine 540, Methyl-coumarin, MitoTracker Red, N,N′-Difluoroboryl-1,9-dimethyl-5-(4-iodophenyl)-dipyrrin, N,N′-Difluoroboryl-1,9-dimethyl-5-[(4-(2-trimethylsilylethynyl), N,N′-Difluoroboryl-1,9-dimethyl-5-phenydipyrrin, Naphthalene, Nile Blue, Nile Red, Octaethylporphyrin, Oregon green, Oxacarbocyanine (C3)dye, Oxadicarbocyanine (C5)dye, Oxatricarbocyanine (C7)dye, Oxazine 1, Oxazine 170, p-Quaterphenyl, p-Terphenyl, Pacific Blue®, Peridinin chlorophyll protein complex (PerCP), Perylene, Phenol, Phenylalanine, Phthalocyanine (Pc), Pinacyanol iodide, Piroxicam, POPOP, Porphin, Proflavin, Propidium iodide, Pyrene, Pyronin Y, Pyrrole, Quinine sulfate, R-Phycoerythrin (PE), Rhodamine, Rhodamine 123, Rhodamine 6G, Riboflavin, Rose bengal, SNARF®, Squarylium dye III, Stains-all, Stilbene, Sulforhodamine 101, SYTOX Blue, TAMRA, Tetra-t-butylazaporphine, Tetra-t-butylnaphthalocyanine, Tetrakis(2,6-dichlorophenyl)porphyrin, Tetrakis(o-aminophenyl)porphyrin, Tetramesitylporphyrin (TMP), tetramethylrhodamine, Tetraphenylporphyrin (TPP), Texas Red® (TR), Thiacarbocyanine (C3)dye, Thiadicarbocyanine (C5)dye, Thiatricarbocyanine (C7)dye, Thiazole Orange, Thymine, TO-PRO®-3, Toluene, TOTO-3, TR, Tris(2,2′-bipyridyl)ruthenium(II), TRITC, TRP, Tryptophan, Tyrosine, Uracil, Vitamin B12, YO-PRO-1, YOYO-1, Zinc octaethylporphyrin (ZnOEP), Zinc phthalocyanine (ZnPc), Zinc tetramesitylporphyrin (ZnTMP), Zinc tetramesitylporphyrin radical cation, and Zinc tetraphenylporphyrin (ZnTPP). Suitable optical dyes are well-known in the art and described in the 1996 Molecular Probes Handbook by Richard P. Haugland.

In various embodiments, the dye may be an Alexa Fluor® dye, including Alexa Fluor® 350, Alexa Fluor® 405, Alexa Fluor® 430, Alexa Fluor® 488, Alexa Fluor® 500, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 647, Alexa Fluor® 660, Alexa Fluor® 680, Alexa Fluor® 700, and Alexa Fluor® 750 (Life Technologies Corporation, 5791 Van Allen Way, Carlsbad, Calif. 92008).

In various embodiments, the dye may be a tandem fluorophore conjugate, including Cy5-PE, Cy5.5-PE, Cy7-PE, Cy5.5-APC, Cy7-APC, Cy5.5-PerCP, Alexa Fluor® 610-PE, Alexa Fluor® 700-APC, and Texas Red-PE. Tandem conjugates are less stable than monomeric fluorophores, so comparing a detection reagent labeled with a tandem conjugate to reference solutions may yield MESF calibration constants with less precision than if a monomeric fluorophore had been used.

In various embodiments, the dye may be a fluorescent protein such as green fluorescent protein (GFP; Chalfie, et al., Science 263(5148):802-805 (Feb. 11, 1994); and EGFP; Clontech-Genbank Accession Number U55762), blue fluorescent protein (BFP; 1. Quantum Biotechnologies, Inc. 1801 de Maisonneuve Blvd. West, 8th Floor, Montreal (Quebec) Canada H3H 1J9; 2. Stauber, R. H. Biotechniques 24(3):462-471 (1998); 3. Heim, R. and Tsien, R. Y. Curr. Biol. 6:178-182 (1996)), cyan fluorescent protein (CFP), and enhanced yellow fluorescent protein (EYFP; 1. Clontech Laboratories, Inc., 1020 East Meadow Circle, Palo Alto, Calif. 94303). In some embodiments, the dye is dTomato, FlAsH, mBanana, mCherry, mHoneydew, mOrange, mPlum, mStrawberry, mTangerine, ReAsH, Sapphire, mKO, mCitrine, Cerulean, Ypet, tdTomato, Emerald, or T-Sapphire (Shaner et al., Nature Methods, 2(12):905-9. (2005)).

In various embodiments, the dye may be a fluorescent semiconductor nanocrystal particle, or quantum dot, including Qdot® 525 nanocrystals, Qdot® 565 nanocrystals, Qdot® 585 nanocrystals, Qdot® 605 nanocrystals, Qdot® 655 nanocrystals, Qdot® 705 nanocrystals, Qdot® 800 nanocrystals (Life Technologies Corporation, 5791 Van Allen Way, Carlsbad, Calif 92008). In some embodiments, the dye may be an upconversion nanocrystal, as described in Wang et al., Chem. Soc. Rev., 38:976-989 (2009).

In various embodiments, the dye may be an ATTO 390 dye, ATTO 425 dye, ATTO 465 dye, ATTO 488 dye, ATTO 495 dye, ATTO 520 dye, ATTO 532 dye, ATTO 550 dye, ATTO 565 dye, ATTO 590 dye, ATTO 594 dye, ATTO 610 dye, ATTO 611X dye, ATTO 620 dye, ATTO 633 dye, ATTO 635 dye, ATTO 637 dye, ATTO 647 dye, ATTO 647N dye, ATTO 655 dye, ATTO 665 dye, ATTO 680 dye, ATTO 700 dye, ATTO 725 dye and ATTO 740 dye manufactured by ATTO-TEC GmbH (Siegen, Germany).

The term “radionuclide”, as used herein, relates to medically useful radionuclides, including, for example, positively charged ions of radiometals such as Y, In, Cu, Lu, Tc, Re, Co, Fe and the like, such as ⁹⁰Y, ¹¹¹In, ⁶⁷-Cu, ⁷⁷Lu, ⁹⁹Tc and the like, preferably trivalent cations, such as ⁹⁰Y and ¹¹¹In.

The term “pharmaceutical agent”, as used herein, encompasses all classes of chemical compounds exerting an effect in a biological system. Preferred pharmaceutical agents for the use in the present invention are molecules selected from the group consisting of DNA, FNA, oligonucleotides, polypeptides, peptides, antineoplastic agents, hormones, vitamins, enzymes, antivirals, antibiotics, antiinflammatories, antiprotozoans, antirheumatics, radioactive compounds, antibodies, prodrugs, and combinations thereof.

“(Bio)Therapeutic agent,” “drug” or “active agent”, as used herein, means any compound useful for therapeutic or diagnostic purposes. The terms as used herein are understood to mean any compound that is administered to a patient for the treatment of a condition that can traverse a cell membrane when attached to a ligand compound of the disclosure.

Therapeutic agents include but are not limited to hydrophilic and hydrophobic compounds. Accordingly, therapeutic agents contemplated by the present disclosure include without limitation drug-like molecules, proteins, peptides, antibodies, antibody fragments, aptamers and small molecules.

Protein therapeutic agents include, without limitation peptides, enzymes, structural proteins, receptors and other cellular or circulating proteins as well as fragments and derivatives thereof, the aberrant expression of which gives rise to one or more disorders. Therapeutic agents also include, as one specific embodiment, chemotherapeutic agents. Therapeutic agents also include, in various embodiments, a radioactive material.

A “chemotherapeutic agent” or “chemotherapeutic drug” is any chemical compound used in the treatment of a proliferative disorder. Examples of chemotherapeutic agents include, without being limited to, the following classes of agents: nitrogen mustards, e. g. cyclophosphamide, trofosfamide, ifosfamide and chlorambucil; nitroso ureas, e. g. carmustine (BCNU), lomustine (CCNU), semustine (methyl CCNU) and nimustine (ACNU); ethylene imines and methyl-melamines, e. g. thiotepa; folic acid analogs, e. g. methotrexate; pyrimidine analogs, e. g. 5-fluorouracil and cytarabine; purine analogs, e. g. mercaptopurine and azathioprine; vinca alkaloids, e. g. vinblastine, vincristine and vindesine; epipodophyllotoxins, e. g. etoposide and teniposide; antibiotics, e. g. dactinomycin, daunorubicin, doxorubicin, epirubicin, bleomycin a2, mitomycin c and mitoxantrone; estrogens, e. g. eiethyl stilbestrol; gonadotropin-releasing hormone analogs, e. g. leuprolide, buserelin and goserelin; antiestrogens, e. g. tamoxifen and aminoglutethimide; androgens, e. g. testolactone and drostanolonproprionate; platinates, e. g. cisplatin and carboplatin; and interferons, including interferon-alpha, beta and gamma.

The chemotherapeutic agents of the present invention are preferably small chemical compounds. Thus, the chemotherapeutic agent has a molecular weight of preferably less than about 5,000, more preferably less than about 3,000, still more preferably less than about 2,000, and most preferably less than about 1,000.

A “platinate” is a chemotherapeutic drug that contains platinum as a central atom. Examples of platinates include cisplatin, carboplatin, oxaliplatin, ormaplatin, iproplatin, enloplatin, nedaplatin, ZD0473 (cis-aminedichloro(2-methylpyridine)-platinum (II)), BBR3464 and the like.

The term “radiotherapeutic agent”, as used herein, is intended to refer to any radiotherapeutic agent known to one of skill in the art to be effective to treat or ameliorate a hyperproliferative disorder, without limitation. For instance, the radiotherapeutic agent can be an agent such as those administered in brachytherapy or radionuclide therapy.

Also encompassed are embodiments, wherein (a) the compound having the structure of formula (I)

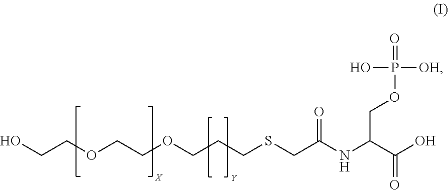

wherein X and Y are independently from each other an integer ranging from 1-10; (b) the ligand compound is selected from the group consisting of H-Ser-(PO₃H₂)—NH-PEG₄-ol, PO₃H₂—O—CH₂—CO-Gly-NH-PEG₄-OH, PO₃H₂—O—CH₂—CO-Ser(PO₃H₂)—NH-PEG₄-OH, PO₃H₂—O—CH₂—CO-Ser(PO₃H₂)—NH-PEG₄-ol, H-Ser-(PO₃H₂)-Ser-Ser-Ser-Ser-ol, H-Ser-(PO₃H₂)-Val-Val-Val-Thr-ol and PO₃H₂—O—CH₂—CO-Ser(PO₃H₂)-Val-Val-Val-Thr-ol.

The term “PEG”, as used herein, means polyethylene glycol. The term “ol”, as used herein, means that the C-terminal carboxylic acid of a given peptide has been reduced to an alcoholic group. The terms “Ser”, “Gly”, “Val”, “Phe”, “Thr”, Tyr“, as used herein, refer to the amino acids serine, glycerine, valine, phenylalanine, threonine and tyrosine, respectively, or their peptide conjugated derivatives.

In more preferred embodiments, X is 3 and Y is 9.

In various embodiments of the invention, the ligand compound has a structure selected from the group consisting of

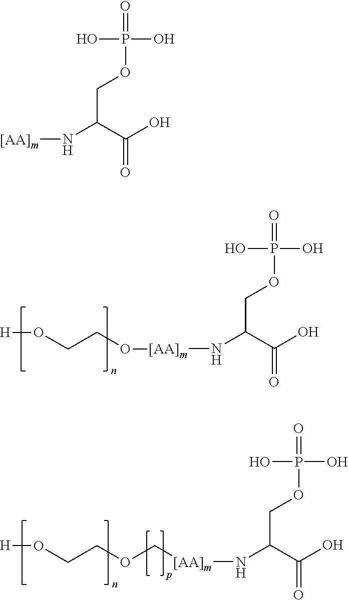

wherein AA represents an amino acid, m is 1-10, n is 1-13 and p is 1-26.

In preferred embodiments, m is 2-8, 3-7 or 4-6. In other preferred embodiments n is 2-10, 3-8 or 4-6. In still other preferred embodiments p is 2-20, 3-15, 4-10 or 5-7.

In a further aspect, the present invention relates to a coated metal nanoparticle comprising a core metal nanoparticle that is coated with a plurality of ligand compounds of the invention.

The term “coating”, as used herein, refers to a process for covering or surrounding a single particle with one or more layers of a coat forming material to stabilize the particle. The term “coated”, as used herein, has a somewhat different meaning compared to “coating” and refers to a single or individual particle which is covered with or surrounded by a coat forming material, wherein the coat forming material remains distinct from the single particle that it covers, and with whose aid the particle is stabilized. While the covering by the coat forming material does not necessarily need to be uniform or to cover or surround the entire particle surface, the covering by the coat forming material should be sufficient to impart improved stability. Preferably, but not necessarily, the coat forming material will completely cover or encase the particle in a substantially uniform layer. In the present invention, the ligand compound represents the coat forming material, while the core metal nanoparticle is covered.

In embodiments of the present invention, the nanoparticles have a size such that they remain suspended or dispersed in a liquid or solution (without agitation), rather than settling under the influence of gravity (disregarding settling due to agglomeration). For spherical nanoparticles, in liquids having a viscosity and density about that of water, that size is typically no greater than about 1000, 500, 400, 300, 200 or 100 nm. In other embodiments, the size of nanoparticles is less than about 50, 40, 30, 20 or 10 nm. In certain other embodiments, the size of nanoparticles is less than about 6 nm. Unless noted otherwise, all references to size set forth herein are the average size of a multiplicity of nanoparticles.

As is known in the art, any of numerous materials may be used to prepare the nanoparticles. Kotov (Nanoparticle Assemblies and Structures, CRC Press 2006.) provides a review of methods and materials for making nanoparticles. The selection of materials for making nanoparticles may depend on the desired property. For example, certain metals, alloys, and oxides are known to have magnetic (ferromagnetic, paramagnetic, superparamagnetic) properties. Examples of magnetic materials comprise chromium (III), cobalt (II), copper (II), dysprosium (III), erbium (III), gadolinium (III), holmium (III), iron (III), iron (II), manganese (II), manganese (III), nickel (II), neodymium (III), praseodymium (III), samarium (III), terbium (III), and ytterbium (III). When sufficiently small, nanoparticles of ferromagnetic material tend to become superparamagnetic (i.e., their magnetic domains cannot be permanently aligned in any particular direction). Ferromagnetic materials, such as alloys of iron and platinum, have high coercivity. Certain semiconductor materials, such as cadmium selenide, cadmium tellurium, cadmium sulfide, zinc sulfide, zinc selenide, lead sulfide, lead selenide, gallium arsenide, gallium phosphide, indium phosphide and indium arsenide are known to have useful electronic or optical properties (such as fluorescence).

In various embodiments, the core metal nanoparticle may comprise or consist of platinum (Pt) or lead (Pb).

In one embodiment of the present invention, the nanoparticles comprise a core that comprises one or more oxides of iron known to be paramagnetic (e.g., magnetite, Fe₃O₄ (which is sometimes represented as FeO.Fe₂O₃), or maghemite, Fe₂O₃). In another embodiment, the core consists essentially of one or more iron oxides such that any other elements present are at what is considered to be impurity levels (e.g., less than about 1 wt %).

In addition to metal, the core may also comprise other materials such as a fluorescent group, a radioactive nuclide, an additional magnetic material, a neutron capture agent, or a combination thereof.

In one embodiment, the core further comprises one or more fluorescent groups. Exemplary fluorescent groups include rhodamine, pyrene, fluorescein and other dyes listed in The Molecular Probes® Handbook—A Guide to Fluorescent Probes and Labeling Technologies 11th edition published by Invitrogen Inc. Compounds comprising these fluorescent groups may be introduced into a solution comprising solute iron and co-precipitated with the iron oxide or they added to the surface of the nanoparticles post synthesis.

In one embodiment the core further comprises one or more magnetic materials that comprise an element selected from the group consisting of aluminum, cerium(IV), chromium(III), cobalt(II), copper(II), dysprosium, erbium, gadolinium, holmium, manganese(II), nickel(II), neodymium, praseodymium(III), samarium(III), ytterbium(III), terbium(III), titanium(IV), yttrium, zirconium, and combinations thereof. These elements may be co-precipitated with the aforementioned metal when forming the core and will typically be in the form of oxides as well.

In one embodiment, the nanoparticle core comprises one or more radioactive materials that are not magnetic. For example, metals may be coprecipitated with radioactive isotopes, such as technetium-99m (U.S. Pat. No. 5,362,473), which may be useful for using the nanoparticles in conducting lung scintigraphy and radiotherapy. Exemplary radionuclides that may be incorporated in the nanoparticle, preferably in the core, include one or more of the following: ¹¹¹Ag, ¹⁹⁹Au, ⁶⁷Cu, ⁶⁴Cu, ¹⁶⁵_{Dy,} ¹⁶⁶_{Dy,} ⁶⁹_{Er,} ¹⁶⁶Ho, ¹¹¹In, ¹⁷⁷Lu, ¹⁴⁰La, ³²P, ¹⁰³Pd, ¹⁴⁹Pm, ¹⁹³Pt, ¹⁹⁵Pt, ¹⁸⁶Re, ¹⁸⁸Re, ¹⁰⁵Rh, ⁹⁰Sr, ¹⁵³Sm, ¹⁷⁵Yb, and ⁹⁰Y.

In various embodiments of the invention, the plurality of ligand compounds of the invention comprises a mixture of at least two structurally different ligand compounds. The term “mixture of at least two structurally different ligand compounds”, as used herein, refers to a combination of two or more ligand compounds of the invention as defined above, wherein said ligand compounds differ from each other in their chemical composition in at least one position. In more preferred embodiments, the difference is based on a distinguishable counter ion in a salt bond but is a difference can still be detected after solution of the ligand compounds in water or in another solvent.

The term “plurality”, as used herein, defined as two or more than two.

The scope of the present invention also encompasses various embodiments wherein the core metal nanoparticle is a metal oxide nanoparticle, preferably iron oxide nanoparticle and more preferably a superparamagnetic iron oxide nanoparticle (SPION).

In certain preferred embodiments, the metal oxide comprises or consists of iron oxide, which in nanoscale form is known as superparamagnetic iron oxide (SPIO). The iron oxide may be in the form of magnetite (Fe₃O₄) or haematite (Fe₂O₃). In other variants, the iron oxide may be mixed with tin oxide, an advantage of which is that a mixture of iron and tin oxide provides contrast for X-Ray as well as magnetic resonance imaging. An exemplary ratio of iron to tin in such a mixture is 2 parts iron to 1 part tin by atomic weight, i.e. in a stoichiometric ratio Fe₂SnO₄. Other metal oxides may alternatively be used.

In a still further aspect of the invention, the scope encompasses the use of a ligand compound of the invention for coating a metal nanoparticle.

In a fourth aspect, the present invention relates to a method of producing a coated metal nanoparticle of the invention comprising: (a) providing a core metal nanoparticle and a plurality of ligand compounds of the invention; and (b) combining the core metal nanoparticle and the plurality of ligand compounds under conditions that allow the formation of the coated metal nanoparticle of the invention.

In various embodiments, the above method comprises prior to step (a) encapsulation of the metal nanoparticle with an intermediate hydrophilic ligand, preferably tetramethylammonium hydroxide (TMAOH).

The term “combining”, as used herein, is intended to mean a mixing or contacting of the ligand compound of the invention and the core metal nanoparticle so that a mixed solution can occur.

Conditions that allow the formation of the coated metal nanoparticle are well-known to the skilled person. In addition, based on the provided examples of this invention the skilled person may modify the conditions to increase parameters, such as reaction time, ratios of the reacted compounds, etc.

In a further aspect, the invention relates to coated metal nanoparticle of the invention for use as a medicament.

The term “medicament”, as used herein, is meant to mean and include any substance (i.e., compound or composition of matter) which, when administered to an organism (human or animal) induces a desired pharmacologic and/or physiologic effect by local and/or systemic action. The term therefore encompasses substances traditionally regarded as actives, drugs and bioactive agents, as well as biopharmaceuticals (e.g., peptides, hormones, nucleic acids, gene constructs, etc.) typically employed to treat a number of conditions which is defined broadly to encompass diseases, disorders, infections, and the like. The coated metal nanoparticles may be used in combination with further agents including, without limitation, antibiotics, antivirals, H₂-receptor antagonists, 5HTₜ agonists, 5HT₃ antagonists, COX2-inhibitors, medicaments used in treating psychiatric conditions such as depression, anxiety, bipolar condition, tranquilizers , medicaments used in treating metabolic conditions, anticancer medicaments, medicaments used in treating neurological conditions such as epilepsy and Parkinsons Disease, medicaments used in treating cardiovascular conditions, non-steroidal anti-inflammatory medicaments, medicaments used in treating Central Nervous System conditions, and medicaments employed in treating hepatitis. The term medicament also encompasses pharmaceutically acceptable salts, esters, solvates, and/or hydrates of the pharmaceutically active substances referred to hereinabove. Various combinations of any of the above medicaments may also be employed.

In various embodiments of the coated metal nanoparticle for use, the ligand compound is functionalized by the attachment of an additional group.

The scope of the present invention also encompasses various embodiments wherein the additional group is selected from the group consisting of a dye, a radionuclide, a pharmaceutical agent, a biotherapeutic agent, a chemotherapeutic agent, a radiotherapeutic agent, and combinations thereof.

In a sixth aspect, the invention relates to a method of producing the ligand compound of the invention comprising:

a) reacting a compound of formula (II)

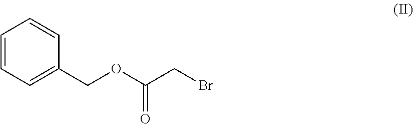 [SMILES: O=C(CBr)OCc1ccccc1]

with NaI to form a compound of formula (III)

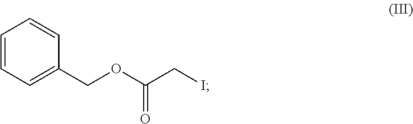 [SMILES: O=C(CI)OCc1ccccc1]

b) reacting the compound of formula (III) with a compound of formula (IV)

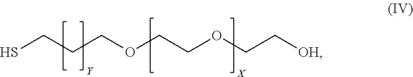

wherein X and Y are independently from each other an integer ranging from 1-10, to form a compound of formula (V)

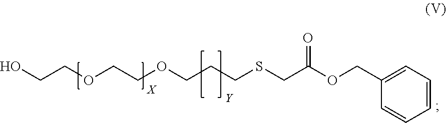

c) reacting the compound of formula (V) with Boc2O to form a compound formula (VI)

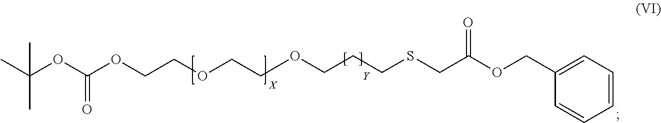

d) reacting the compound of formula (VI) with H₂ to form the a compound of formula (VII)

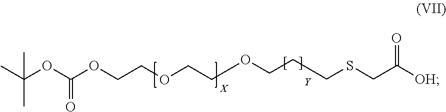

e) reacting the compound of formula (VII) with a compound of formula (VIII)

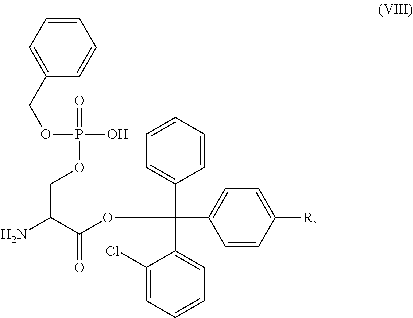

wherein R represents a resin,
to form a compound of formula (IX)

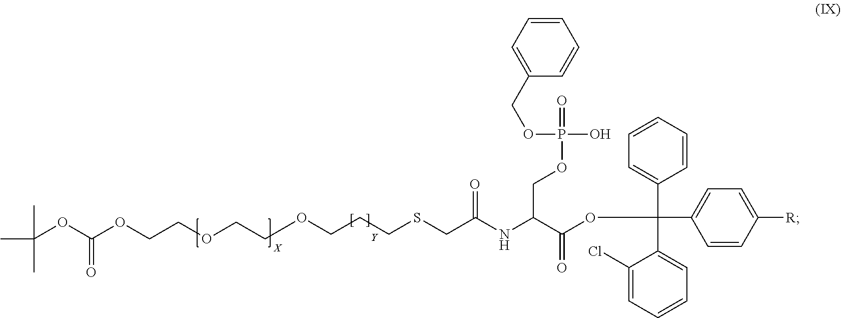

f) reacting the compound of formula (IX) with dichloromethane (DCM): triflouroacetic acid (TFA) to form a compound of formula (X)

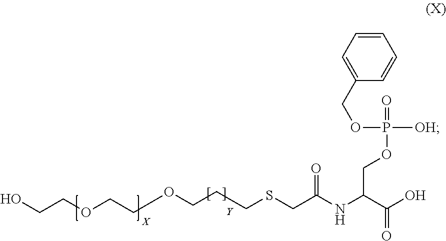

and
g) reacting the compound of formula (X) with H₂ to form the ligand compound of the invention.

The term “reacting”, as used herein, refers to a chemical process or processes in which two or more reactants are allowed to come into contact with each other to effect a chemical change or transformation. For example, when reactant A and reactant B are allowed to come into contact with each other to afford a new chemical compound(s) C, A is said to have “reacted” with B to produce C.

“At least one”, as used herein, relates to one or more, in particular 1, 2, 3, 4, 5, 6, 7, 8, 9, 10 or more.

As used herein, the terms “approximately”, “about” or “ca.”, as applied to one or more values of interest, refer to a value that is similar to a stated reference value. In certain embodiments, the terms “approximately”, “about” or “ca.” refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less of the stated reference value.

### EXAMPLES

### Methods and Materials

### Materials

Sodium iodide, benzyl bromoacetate, acetone, diethyl ether, sodium hydrogen carbonate, acetonitrile, ethyl acetate, magnesium sulphate, di-tert-butyl dicarbonate (Boc₂O), 4-(dimethylamino) pyridine (DMAP), dichloromethane (DCM), petroleum ether, ethanol, dimethylformamide (DMF), thionyl chloride, Fmoc-O-(benzylphospho)-L-serine (Fmoc-Ser(PO₃BzlH)—OH), N,N-diisopropylethylamine (DIEA), piperidine, O-(benzotriazol-1-yl)-N,N,N′,N′-tetramethyluronium hexafluorophosphate (HBTU), 4-methylmorpholine (NMM), trifluoroacetic acid (TFA), ascorbic acid, trisodium citrate dihydrate, ammonium acetate and 3-(2-pyridyl)-5,6-di(2-furyl)-1,2,4-triazine-5′,5″-disulfonic acid disodium salt (ferrozine) were all purchased from Sigma-Aldrich (Dorset, UK) at the highest purity and used without further purification. The EG alkanethiol ligand, HS-(CH₂)₁₁-EG₄-OH, was purchased from Prochimia (ProChimia Surfaces Sp. z o.o, Sopot, Poland). Palladium hydroxide (99% purity) was purchased from Alfa Aesar (Heysham, Lancashire, UK). Trityl chloride resin (Trt-Cl resin, 100-200 mesh, 1% (w/v) divinyl benzene (DVB) crosslinking, 1.0-2.0 mmol/g loading) was purchased from Iris Biotech (Marktredwitz, Germany). SPIONs (8.5 nm diameter) coated in oleic acid and soluble in toluene, prepared as described (Park et al. 2004), were a gift from Anita Peacock (Department of Chemistry, University of Liverpool). Nanosep centrifugal ultrafiltration devices (10 kDa) were purchased from PALL (PALL Corp., Portsmouth, Hants, UK). Sephadex G-25 superfine, diethylaminoethyl (DEAE) Sepharose Fast Flow and carboxymethyl (CM) Sepharose Fast Flow were purchased from GE Healthcare (Little Chalfont, Bucks, UK).

### Exchange of Oleic Acid Ligand on SPIONs for EG Alkanethiol Phosphoserine

SPIONs were diluted to 5 mg/mL with toluene and 500 KL placed in a 10 kDa Nanosep centrifugal filtration unit. They were concentrated to 100 KL by centrifuging at 9000 g at 4° C., with the filtration unit being changed if it showed signs of swelling due to long contact with the toluene. The SPIONs were then made back up to 500 KL with toluene before being concentrated back down to 100 KL. This was repeated a further two times to give four toluene washes in total. After the final centrifugation, SPIONs were made up to 500 KL in toluene (5 mg/mL) and were then diluted 1:40 in THF and vortexed for 1 min. From a 10 mM stock in ethanol, the EG alkanethiol phosphoserine ligand was diluted to 2 mM with 150 mM NaCl in deionised water. One volume of this solution was slowly added to the SPIONs dropwise, vortexing well between additions. This was then left to react overnight at 4° C. and then for 4 h at room temperature the following day. The SPIONs were centrifuged for 7 min at 11,000 g and the supernatant removed. The pellet was resuspended in deionised water containing 150 mM NaCl and 2 mM EG alkanethiol phosphoserine ligand and was incubated for 48 h at 4° on a rotary mixer. The SPIONs were then concentrated with a Nanosep centrifugal filtration unit and subjected to size exclusion chromatography with Sephadex G25 superfine except with 150 mM NaCl as the mobile phase and equilibration of the column with 0.2 mM EG alkanethiol phosphoserine ligand. SPIONs eluting in the void volume were re-incubated with 0.2 mM EG alkanethiol phosphoserine ligand overnight at 4° C. on a rotary mixer. The following day, excess ligand was separated from the SPIONs using size-exclusion chromatography on Sephadex G25 superfine, with 1× PBS as the mobile phase. Tween-20 was then added to the SPIONS eluting in the void volume give a 0.01% (v/v) final concentration.

### Ion-exchange Chromatography

DEAE or CM Sepharose was added to 10 mL columns to give a volume of 200 μL resin. The resins were equilibrated with 20 column volumes of 1× PBS and then washed with 10 column volumes of water. SPIONs were concentrated to between 10 μL and 50 μL using a 10 kDa Nanosep centrifugal filtration unit and were then resuspended in water. This was repeated three times to remove excess electrolytes. The SPIONs were then loaded onto the columns and the flow-through collected as one fraction. The columns were then washed with 50 μL aliquots of water to remove unbound SPIONs. Eluted SPIONs were collected as one fraction.

### Citrate Assay

A citrate assay was carried out, as previously described to determine the stability of the SPIONs to challenge by a small chelating agent (Arbab et al. 2005, Levy et al. 2010, Soenen et al. 2010). SPIONs (1 Kg) were incubated with 100 KL sodium citrate tribasic (20 mM in PBS) at pH 7.14, 5.5, and 4.5 for up to 9 days at 37° C. in separate wells of a 96 well plate. Ferrozine reagent (30 KL, 6.5 mM ferrozine, 100 mM ascorbic acid and 1M ammonium acetate in deionised water) was then added to each of the wells for 3 h. The absorbance at 595 nm was measured using a SpectraMax Plus384 spectrometer. The amount of iron ions in the solution was then determined by comparing to a calibration curve produced with an iron standard containing between 0 Kg and 1 Kg of iron.

### Example 1

### Synthesis of the EG Alkanethiol Phosphoserine Ligand

The EG alkanethiol phosphoserine ligand for the SPIONs was synthesized using the protocol described below. Where possible, the progress of the reaction was monitored by thin layer chromatography (TLC) and reaction products were characterised by 1H NMR using a Bruker AMX 400 at 400 MHz.

i—Synthesis of Benzyl iodoacetate (1)

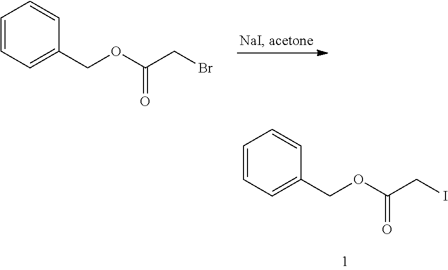 [SMILES: O=C(CBr)OCc1ccccc1 ; O=C(CI)OCc1ccccc1]

Sodium iodide (5 mmol) was added to a solution of benzyl bromoacetate (1 mmol) in acetone (5 mL/mmol) at room temperature and stirred for 2 h. The reaction mixture was diluted with diethyl ether and stirred at room temperature for 20 min before being filtered through celite and concentrated in vacuo. The residue was suspended in diethyl ether and filtered through celite to give the product as an orange oil, which was used without further purification.

1H NMR (400 MHz, CDCl3): 7.39-7.26 (5H, m, 5 x ArH), 5.18 (2H, s, CH₂Ph), 3.82 (2H, s, CH₂I).
ii—Synthesis of Benzyl-1-hydroxy-3,6,9,12-tetraoxa-24-thiahexacosan-26-oate (3)

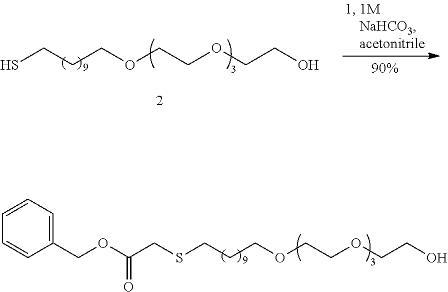

A solution of the EG alkanethiol ligand 2 (1 mmol) and benzyl iodoacetate (1 mmol) in 1 M aqueous sodium hydrogen carbonate (2 mL/mmol) and acetonitrile (4 mL/mmol) was stirred at room temperature for 3 h. The product was extracted with ethyl acetate (3×10 mL) and the combined organics were dried over MgSO₄ then concentrated in vacuo. The crude product was purified using flash column chromatography (SiO₂ eluting with ethyl acetate) to give the product as a pale yellow oil (90% yield).

1H NMR (400 MHz, CDCl3): 7.38-7.32 (5H, m, 5x ArH), 5.17 (2H, s, CH₂Ph), 3.74-3.58 (16H, m, 8x CH₂), 3.45 (2H, t, J 6.9, CH₂), 3.26 (2H, s, CH₂), 2.66-2.58 (3H, m, CH₂ and OH), 1.62-1.25 (18H, m, 9 x CH₂).
iii—Synthesis of Benzyl-2,2-dimethyl-4-oxo-3,5,8,11,14,17-hexaoxa-29-thiahentriacontan-31-oate (4)

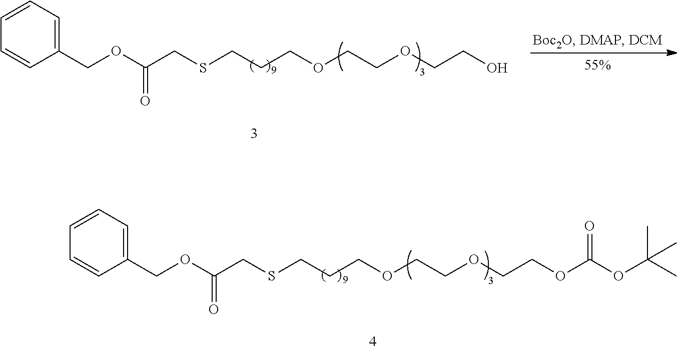

Boc₂O (1.1 mmol) was added to a solution of 3 (1 mmol) and DMAP (0.1 mmol) in DCM (10 mL/mmol) and the resulting solution was stirred at room temperature overnight. The reaction mixture was washed with saturated aqueous NaHCO₃ and water then dried over MgSO₄ and concentrated in vacuo. The crude product was purified by flash column chromatography (SiO₂ eluting with petroleum ether:ethyl acetate 4:1) to give the product as a colourless oil (55% yield).

1H NMR (400 MHz, CDCl3): 7.31-7.27 (5H, m, 5x ArH), 5.10 (2H, s, CH₂Ph), 4.15-4.12 (2H, m, CH₂) 3.65-3.49 (14H, m, 7 x CH₂), 3.37 (2H, t, J 6.8, CH₂), 3.18 (2H, s, CH₂), 2.53 (2H, t, J 7.4, CH₂), 1.58-1.22 (27H, m, 9x CH₂ and 3x CH3).
iv—Synthesis of 2,2-Dimethyl-4-oxo-3,5,8,11,14,17-hexaoxa-29-thiahentriacontan-31-oic acid (5)

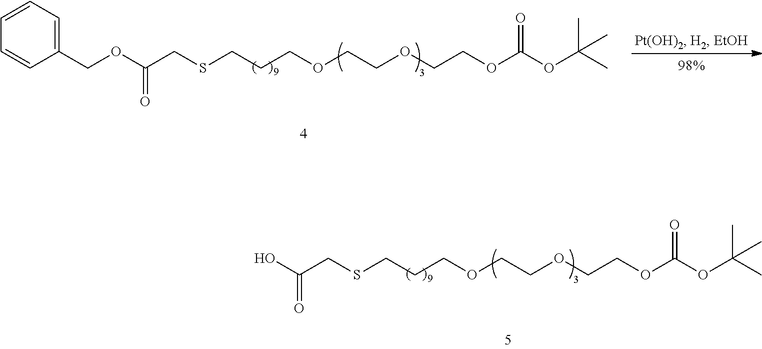

Palladium hydroxide (0.1 mmol) was added to a solution of 4 (1 mmol) in ethanol (20 mL/mmol). The reaction mixture was evacuated and back-filled with hydrogen three times and then stirred under an atmosphere of hydrogen for 4 h. The reaction mixture was filtered through a pad of celite and then concentrated in vacuo to give the product as a colourless oil (98% yield).

1H NMR (400 MHz, CDCl3): 11.46 (1H, brs, OH), 4.18-4.16 (2H, m, CH₂) 3.65-3.40 (16H, m, 8x CH₂), 3.23 (2H, s, CH₂), 2.64 (2H, t, J 7.3, CH₂), 1.57-1.22 (27H, m, 9x CH₂ and 3x CH3).
v—Pre-activation of Trt-Cl Resin

The Trt-Cl resin was pre-activated, as described (Harre et al. 1999), by adding thionyl chloride 1.7% (v/v) in DCM and stirring for an hour at room temperature. The resin was then filtered and washed with DMF once and then with DCM three times before being vacuum dried.

vi—Coupling of Fmoc-O-(benzylphospho)-L-serine to Trt-Cl resin (6)

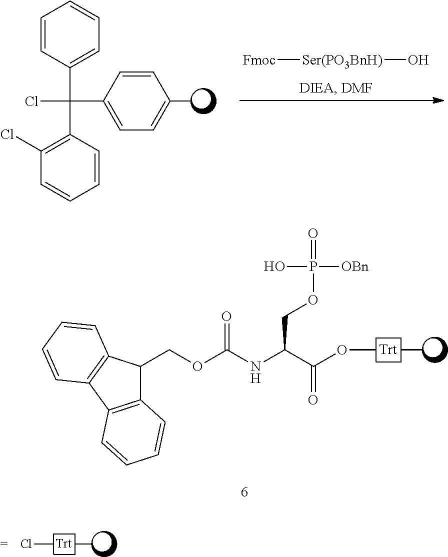

Fmoc-O-(benzylphospho)-L-serine was conjugated to the activated Trt-Cl resin adding the Fmocserine-phosphate 2 mmol) and DIEA (2 mmol) to the dried resin (1 mmol) in DMF (10 mL/mmol) and then stirring at room temperature overnight. The resin was filtered and washed sequentially with DMF and diethyl ether then dried under vacuum.

vii—Fmoc deprotection of Fmoc-O-(benzylphospho)-L-serine on Trt-Cl resin (7)

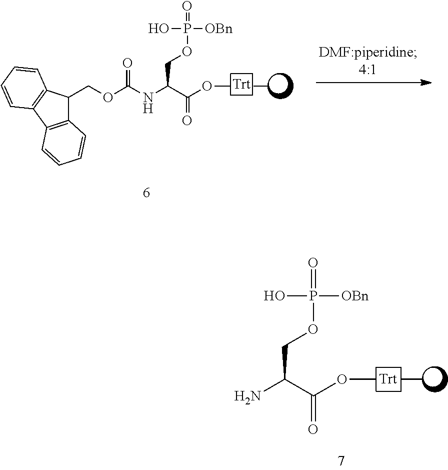

The amine of the Fmoc-O-(benzylphospho)-L-serine was deprotected by adding piperidine 20% (v/v) in DMF (10 mL/mmol) to the dried resin (1 mmol). The reaction was stirred at room temperature overnight. The resin was then filtered and washed sequentially with DMF and then diethyl ether then dried under vacuum.

viii—Coupling of the compound (4) to phosphoserine on Trt-Cl resin (8)

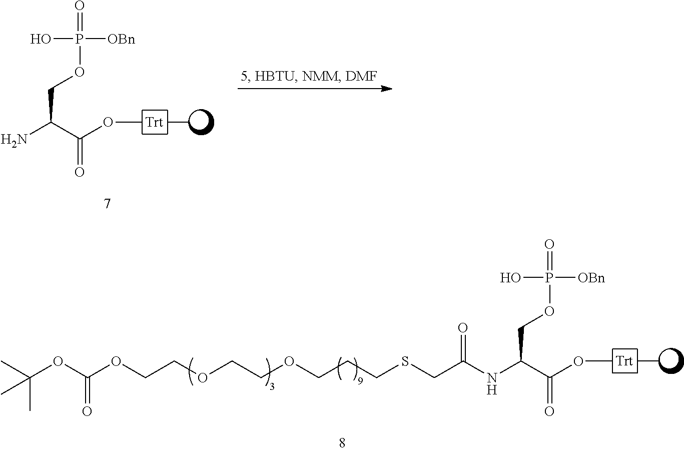

The Boc protected EG ligand 5 was then conjugated to the amine of the phosphoserine. Activation of the carboxylic acid of 5 (1 mmol) was performed by adding HBTU (2 mmol) in DMF (5 mL/mmol) followed by NMM (2 mmol). This mixture was then stirred at room temperature for 30 min. This was then transferred to second vial containing the resin (0.5 mmol) in DMF (5 mL/mmol) and the reaction mixture was stirred at room temperature overnight. The resin was collected by filtration and washed with DMF then water to remove the urea by-product. The resin was then washed sequentially with DMF and then diethyl ether before being vacuum dried.

ix—Acid Cleavage of the Trt-Cl Resin to Prepare the (28S)-28-((((benzyloxy)(hydroxy)phosphoryl)oxy)methyl)-1-hydroxy-26-oxo-3,6,9,12-tetraoxa-24-thia-27-azanonacosan-29-oic acid (9)

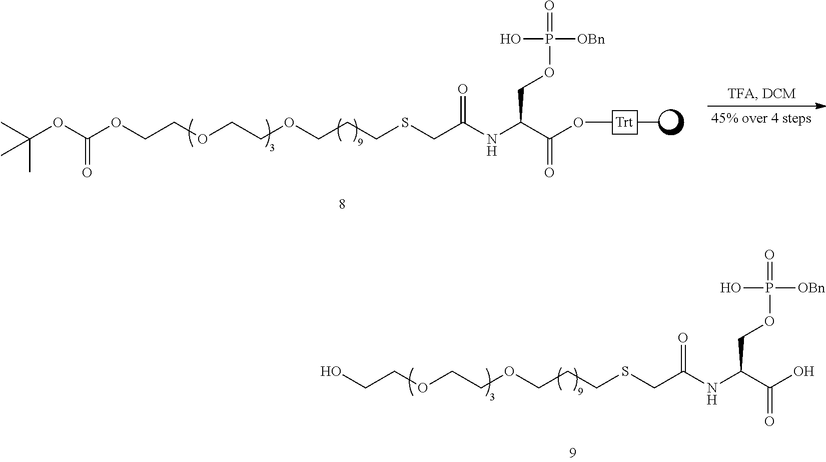

Cleavage from the resin was achieved using DCM:TFA (10:1) which also removed the Boc protecting group yielding 9 in an overall yield of 45%.

1H NMR (400 MHz, d-6-DMSO): 10.55 (2H, brs, 2x OH), 7.45-7.38 (5H, m, 5x ArH), 5.36-5.32 (2H, m, CH₂), 4.44-4.41 (1H, m, 1 of CH₂), 3.65-3.40 (22H, m, 10x CH_{2,} CH and one of CH₂), 3.18 (2H, s, CH₂), 2.58 (2H, t, J 7.4, CH₂), 1.54-1.22 (18H, m, 9x CH₂).
x—Synthesis of (S)-1-hydroxy-26-oxo-28-((phosphonooxy)methyl)-3,6,9,12-tetraoxa-24-thia-27-azanonacosan-29-oic acid (EG alkanethiol phosphoserine) (10)

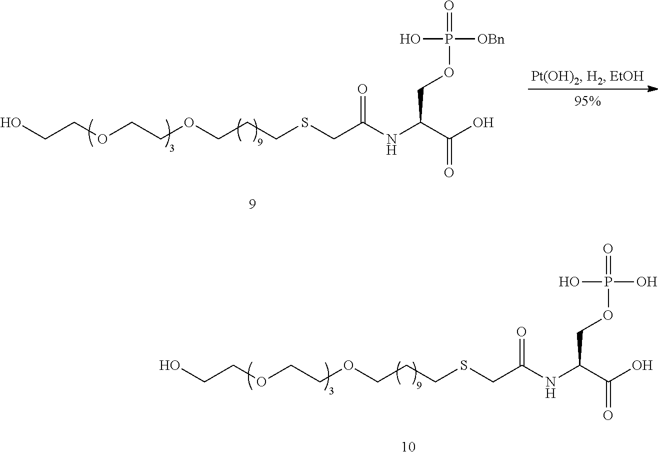

Palladium hydroxide (0.1 mmol) was added to a solution of 9 (1 mmol) in ethanol (10 mL/mmol). The reaction mixture was evacuated and back-filled with hydrogen three times and then stirred under an atmosphere of hydrogen for 4 h. The reaction mixture was filtered through a pad of celite and then concentrated in vacuo to give the product as an off white solid (95% yield).

1H NMR (400 MHz, d-6-DMSO): 11.22 (2H, brs, 2x OH), 10.99 (1H, brs, OH) 4.48-4.43 (1H, m, 1 of CH₂), 3.63-3.42 (22H, m, 10x CH_{2,} CH and one of CH₂), 3.19 (2H, s, CH₂), 2.58-2.55 (2H,m, CH₂), 1.60-1.23 (18H, m, 9x CH₂).

### Example 2

### Ligand-exchange Mediated Transfer of SPIONs to Aqueous Solutions

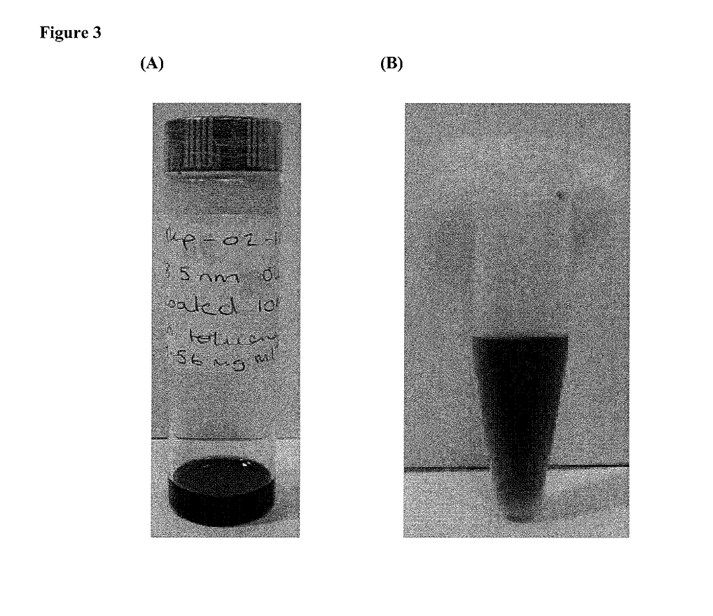

Upon receipt, the SPIONs were coated in oleic acid ligands and were soluble in toluene (FIG. 3A). For these SPIONs to be suitable for biological applications, they first have to undergo ligand exchange to render them soluble in aqueous solutions (FIG. 3B).

The protocol requires THF to act as an intermediate solvent for the ligand-exchange reaction to take place, multiple loadings of the incoming ligand and a ligand-exchange step using Sephadex G25 chromatography equilibrated with the incoming ligand. No chloroform washing as performed during the SPION ligand-exchange protocol, as it was found that performing washes of the SPIONs using toluene before any EG alkanethiol phosphoserine ligand was added was sufficient to remove enough of the outgoing ligand to make efficient ligand exchange possible without destabilising the nanoparticles. When the EG alkanethiol phosphoserine ligand was added, it was added in 150 mM NaCl rather than PBS. The NaCl provided the electrolytes required to drive the packing of the monolayer, but without the phosphate ions present in PBS, which could have competed with the incoming ligand and prevented the formation of a SAM sufficiently robust to impart good colloidal stability. Furthermore, longer incubation times and higher concentrations of incoming ligand were required for ligand-exchange to occur on the SPIONs.

The stability of the water soluble SPIONs against non-specific interactions was tested with biomimetic chromatography resins, Sephadex G25 superfine, DEAE Sepharose and CM Sepharose. Resistance to non-specific binding is, perhaps, even more critical for SPIONs, which have the potential to be used for in vivo imaging. A full understanding of both non-specific interactions of the ligand shell and specific targeting by functional ligands when nanoparticles are transplanted in vivo are almost certainly a prerequisite of nanoparticles gaining regulatory approval.

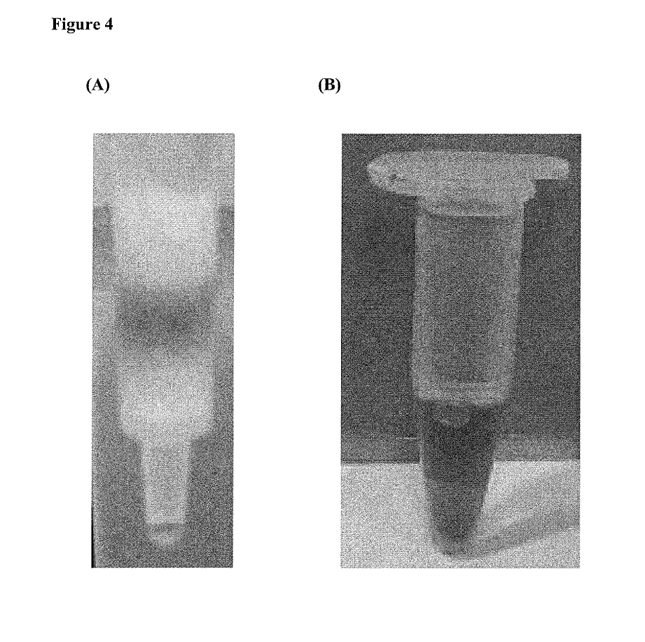

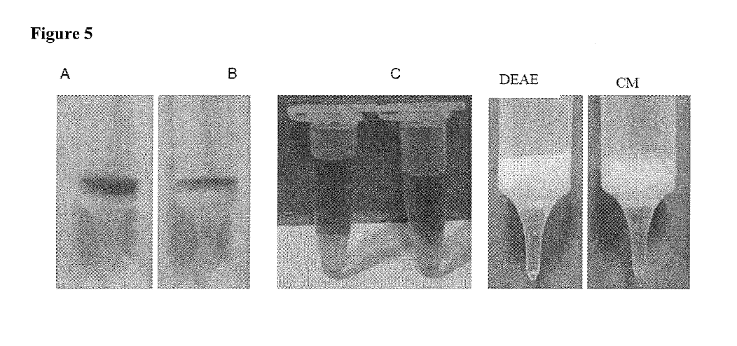

EG alkanethiol phosphoserine capped SPIONs passed through the G25 chromatography resin (FIG. 4A) and were eluted in the excluded volume (FIG. 4B). EG alkanethiol phosphoserine capped SPIONs that eluted at V₀ from the G25 column were subjected to DEAE and CM ion-exchange chromatography (FIG. 5A). All of the SPIONs were eluted from the DEAE and CM resin with water (FIG. 5B) and no SPIONs were detected on the DEAE or CM resin after the water washes (FIG. 5C). This shows that the EG alkanethiol phosphoserine capped SPIONs were neutral in charge and that the EG alkanethiol phosphoserine ligand forms a ligand shell that is sufficiently closely packed to prevent access of the anime and carbonyl groups on these resins from binding to the iron oxide core of the SPIONs.

### Example 3

### Decomposition of SPIONs in the Citrate Assay

EG alkanethiol phosphoserine capped SPIONs that passed through all of the chromatography resins were used in a citrate assay to investigate the decomposition of the core materials of the nanoparticles.

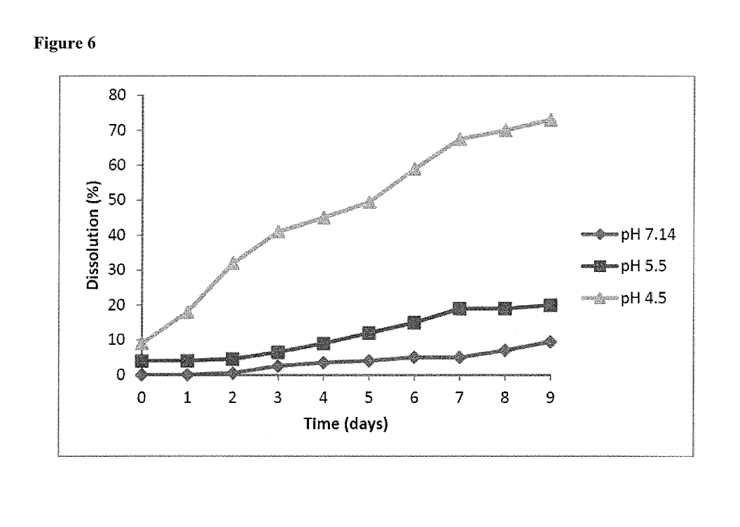

As the pH of the sodium citrate buffer decreased, the dissolution of the SPIONs increased (FIG. 6), showing that the SPIONs decompose much more quickly in acidic environments in the presence of citrate. At pH 7.14 and 5.5 only a small increase in dissolution of the SPION cores was observed over the first two days show. In contrast, at pH 4.5, a more substantial decomposition of the materials was apparent one day after addition of the citrate buffer (FIG. 6).

### Example 3

### Novel Ligands

A novel library of peptides and ligands was designed to prepare peptide coated of iron oxide nanoparticles.

TABLE 1
Peptides and ligands library.

| Names | Structures |
|---|---|
| Ligand L1 | (HO)2-PO-S-C11-EG3-OH |
| Ligand L2 | (HO)2-PO-S-C11-EG6-OH |
| Ligand L3 | (HO)2-PO-S-(CH2)16-OH |
| Peptide S1 | H-Ser(PO3H2)-NH-PEG4-ol |
| Peptide S3 | PO3H2-O-CH2-CO-NH-PEG-ol |
| Peptide S5 | PO3H2-O-CH2-CO-Gly-NH-PEG4-OH |
| Peptide S6 | PO3H2-O-CH2-CO-Gly-NH-PEG4-ol |
| Peptide S7 | PO3H2-O-CH2-CO-Ser(PO3H2)-NH-PEG4-OH |
| Peptide S8 | PO3H2-O-CH2-CO-Ser(PO3H2)-NH-PEG4-ol |
| Peptide S9 | H-Ser(PO3H2)-Ser-Ser-Ser-Ser-ol |
| Peptide S11 | H-Ser(PO3H2)-Phe-Phe-Phe-Thr-ol |
| Peptide S13 | H-Ser(PO3H2)-Val-Val-Val-Thr-ol |
| Peptide S14 | PO3H2-O-CH2-CO-Ser(PO3H2)-Val-Val-Val-Thr-ol |
| Peptide S15 | H-Ser(PO3H2)-C11-PEG4-ol |
| Peptide S16 | PO3H2-O-CH2-CO-Ser(PO3H2)-C11-PEG4-ol |
| Peptide S18 | PO3H2-O-CH2-CO-Tyr(PO3H2)-Val-Val-Val-Thr-ol |

The library currently consists of 16 peptides and ligands. The rationale for the design of the peptides is presented in the Table 2. All peptides and ligands present one or two phosphoric acid moieties at the foot position, allowing the binding to the surface of the iron oxide nanoparticles. The foot may be a phosphorylated amino acid, i.e., phosphoserine or phosphotyrosine, a phosphorothioic acid or a phosphoglycolic acid. The second phosphorylation is conjugated at the N-terminal of a phosphorylated amino acid. The stem is made of a peptide sequence, alkane chain or ethylene glycol of different length. The peptide sequences used here were evaluated previously and showed great potential to prepare highly stable peptide shells on to gold nanoparticles surfaces of the same size (10 nm diameter). The head of the ligands are all hydrophilic and may consist of either a carboxylic acid or alcohol, allowing for water stability and tuning of the charge on the surface of the self-assembled monolayer.

TABLE 2
Design of phosphorylated peptides and ligands for iron oxide nanoparticles coating.

| Design | | | Ligands | | | Peptides | | | | | | | | | | | | |
|---|---|---|---|---|---|---|---|---|---|---|---|---|---|---|---|---|---|---|
| Position | Description | Structure | 1 | 2 | 3 | 1 | 3 | 5 | 6 | 7 | 8 | 9 | 11 | 13 | 14 | 15 | 16 | 18 |
| Foot | Single phosphoric | Phosphoserine | | | | X | | | | X | X | X | X | X | X | X | X | |
| | acid | Phosphotyrosine | | | | | | | | | | | | | | | | X |
| | | Phosphorothioic acid | X | X | X | | | | | | | | | | | | | |
| | | Phosphoglycolic acid | | | | | X | X | X | | | | | | | | | |
| | Double phosphoric | N-terminal phosphorylated | | | | | | | | X | X | | | | X | | X | X |
| | acid | glycolic acid | | | | | | | | | | | | | | | | |
| Stem | Peptide | -Gly- | | | | | | X | X | | | | | | | | | |
| | | -Val-Val-Val-Thr- | | | | | | | | | | | | X | X | | | X |
| | | -Ser-Ser-Ser-Ser- | | | | | | | | | | X | | | | | | |
| | | -Phe-Phe-Phe-Thr- | | | | | | | | | | | X | | | | | |
| | Alkane chain | —(CH2)11— | X | X | | | | | | | | | | | | X | X | |
| | | —(CH2)16— | | | X | | | | | | | | | | | | | |
| | Ethylene glycol | —(CH2—O)3— | | | | X | X | X | X | X | X | | | | | X | X | |
| | | —(CH2—O)3—CH2— | | | | | | | | | | | | | | | | |
| Head | Alcohol | —OH | X | X | X | X | X | | X | | X | X | X | X | X | X | X | X |
| | Carboxylic acid | —CO2H | | | | | | X | | X | | | | | | | | |

All ligands were evaluated for the preparation of self-assembled monolayers on the surface of the iron oxide nanoparticles as presented in the objective 5.

Evaluation of Peptide Ligand Shells for stabilisation of SPIONs.

In this objective, the library of peptide ligands was used and evaluated for the preparation of biocompatible and highly stable peptide coated iron oxide nanoparticles. For all the tests, the oleic acid coated iron oxide nanoparticles (10 nm diameter) from Ocean NanoTech LLC were used.

### Materials, Protocols and Definitions

### Materials

Oleic acid coated iron oxide nanoparticles were purchased from Sigma Aldrich and Ocean NanoTech LLC (ocean) with an average diameter of 5 nm and 10 nm respectively. The peptides and ligands were purchased from ProChimia Surfaces Sp and Peptidesynthetics respectively, and used without further purification. Dimethyl sulfoxide, ferrozine [3-(2-pyridyl)-5,6-diphenyl-1,2,4-triazine-p,p′-disulfonic acid monosodium salt hydrate], tetramethyl ammonium hydroxide, 1-hexadecene, sodium oleate, hexane, 1-octadecene and iron chloride were purchased from Sigma Aldrich. All experiments were conducted using MilliQ water.

### UV-Vis Determination

UV-visible spectra were recorded at room temperature using a Molecular Probes (Oregon, USA) Spectramax 384-well spectrometer, using a 1 cm path length quartz cuvette, and a fixed slit width of 2 nrn. The spectrometer was calibrated daily using the machine's ‘Auto-Calibrate’ air calibration.

### Purification of Peptide Coated Nanoparticles

The peptide coated iron oxide nanoparticles were purified by size-exclusion chromatography using G25 Sephadex resin using H₂O as the solvent. Sephadex G25 superfine (10 mL) columns were stored in H₂O/EtOH. The column was equilibrated with 30mL H₂O. Ligand capped iron oxide nanoparticles (1 mL) were concentrated to 100 μl by centrifugation. The nanoparticles were loaded on the column and eluted under gravity.

### Normalized Aggregation Parameter

To allow the comparison of results from different ligand exchange experiments, the aggregation parameter (AP) was defined as follows:

AP=(A_{460 nm}−A_{ref460 nm})/(A_{300 nm}−A_{ref300 nm})

A_{460 nm} and A_{300 nm} are absorbance values of solutions of nanoparticles at 460 nm and 300 nm, respectively. The empiric wavelength of 460 nm has been chosen to reflect the aggregated state of the nanoparticles. A_{ref460 nm} and A_{ref300 nm} are absorbance values of water at 460 nm and 300 nm. To allow comparison of results obtained with different ligand shells, this primary aggregation parameter was then normalized by dividing the aggregation parameter values of each experiment by the initial aggregation parameter value of the same experiment before the stability test. This provides a Normalized Aggregation Parameter (NAP). A stable sample should have a stable UV-visible absorbance spectrum and hence, its NAP is near 1. An increase of the NAP indicates the instability and eventually aggregation of the nanoparticles.

### Yield of Preparation of Coated Iron Oxide Nanoparticles After Purification

The yield of preparation of peptide coated iron oxide nanoparticles is calculated based on the absorbance at 300 nm of the samples before and after coating and purification by size-exclusion chromatography with G25 resin columns.

Yield=100*(A_{300 after G25}/A_{300 control})

where A_{300 after G25} is the absorbance at 300 nm of the sample after purification with G25 and the total volume of the sample adjusted to 1 mL with water. A_{300 control} is the absorbance at 300 nm of the control sample using only water to obtain the same concentration of iron oxide nanoparticles coated with TMAOH.

### Two Procedures to Prepare Peptide Coated Iron Oxide Nanoparticles

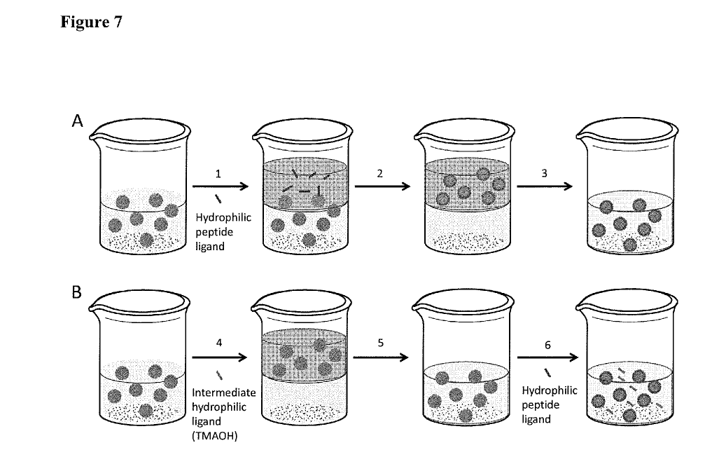

The preparation of water soluble iron oxide nanoparticles using oleic acid coated iron oxide nanoparticles is generally done by ligand exchange of the lipophilic oleic acid ligands with a hydrophilic ligand. There are two major procedures to perform this ligand exchange and obtain peptide coated iron oxide nanoparticles. The first procedure (FIG. 7A) is done by direct transfer using biphasic mixtures of oleic acid coated iron oxide nanoparticles dispersed in organic solvent with an aqueous solutions of peptides. The second method (FIG. 7B) uses an intermediate coating with a hydrophilic ligand, e.g., TMAOH, to transfer the nanoparticles into water. The intermediate coating is than replaced by the peptide coating by ligand exchange in water. The intermediate hydrophilic ligand binds weakly to the iron oxide nanoparticles surface compared to the peptide ligands.

Both methods were evaluated in order to determine the most performing strategy to provide the desired peptide coated iron oxide nanoparticles.

Direct Water Transfer by Ligand Exchange of Oleic Acid Coating with Peptide Ligands

The objective here was to determine the efficiency of a water transfer of the oleic acid coated iron oxide nanoparticles by direct transfer by ligand exchange of oleic acid coating with a hydrophilic phosphorylated from the library. The ligand L1 (Table 1) was used for this study.

Various attempts of water transfer of the nanoparticles were performed using different binary mixtures (toluene/water, chloroform/water) and ternary mixtures (toluene/tetrahydrofuran/water, chloroform/tetrahydrofuran/water) and concentration of aqueous solutions of ligand L1. None of these attempts allowed for an effective production of hydrophilic iron oxide nanoparticles coated with the ligand L1.

The second method of peptide coating of iron oxide nanoparticles was then tested.

Ligand Exchange of Water Soluble Coated Iron Oxide Nanoparticles with Peptide Ligands

In this method, the tetramethylammonium hydroxide (TMAOH) was used as an intermediate hydrophilic ligand to transfer the oleic acid coated nanoparticles in water. This method produced a homogenous and stable solution of TMAOH coated iron oxide nanoparticles. The TMAOH coated nanoparticles were used for all peptide coating experiments.

Water Transfer of Oleic Acid Coated Iron Oxide Nanoparticles with TMAOH Procedure

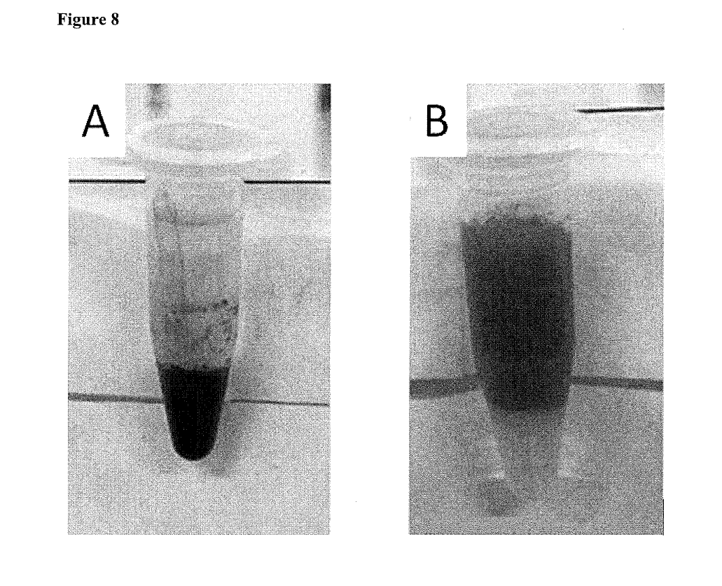

The oleic acid coated nanoparticles (10 nm) (73.8 μl, Fe 15 mg/ml) were diluted in 200 μl of CHCl₃ to produce a solution of Fe 4.05 mg/ml. 1 ml of a 2 mM aqueous solution of TMAOH was then added. The solution was mixed for 30 minutes after which a distinct transfer of the iron oxide nanoparticles from the organic phase to the aqueous phase was observed (FIG. 8). The resultant aqueous phase was extracted and washed 3 times with 200 μl of CHCl₃ prior to removal of excess TMAOH with water by filtration with NanoSep (10 KDa) centrifugal filters. The nanoparticles collected in the filters were re-dissolved in 1.5 ml of H₂O to make up a 10 mM Fe content stock solution of iron oxide nanoparticles.

### Stability Evaluation of TMAOH Coated Iron Oxide Nanoparticles

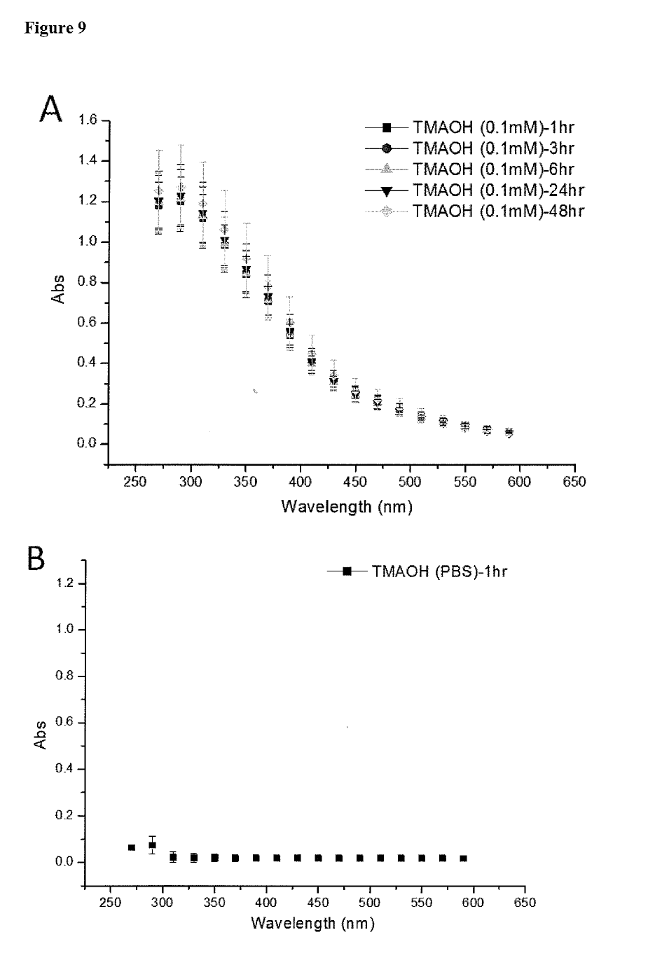

The TMAOH coated iron oxide nanoparticles were diluted to a concentration of 1 mM of iron content prior to the stability test. The stability of the nanoparticles was followed by UV-visible spectrometry. A decrease of absorbance indicates the aggregation of the nanoparticles. The results (FIG. 9) suggest that the TMAOH coated iron oxide nanoparticles are perfectly stable in a 0.1 mM solution of TMAOH, indicating the possibility to store the nanoparticles over a long period of time. Moreover, the TMAOH coated iron oxide nanoparticles rapidly aggregated in the presence of PBS buffer within 1 hour, showing that the TMAOH intermediate hydrophilic coating does not protect the nanoparticles from electrolyte-induced aggregation.

### Non-specific Binding Evaluation of TMAOH Coated Iron Oxide Nanoparticles

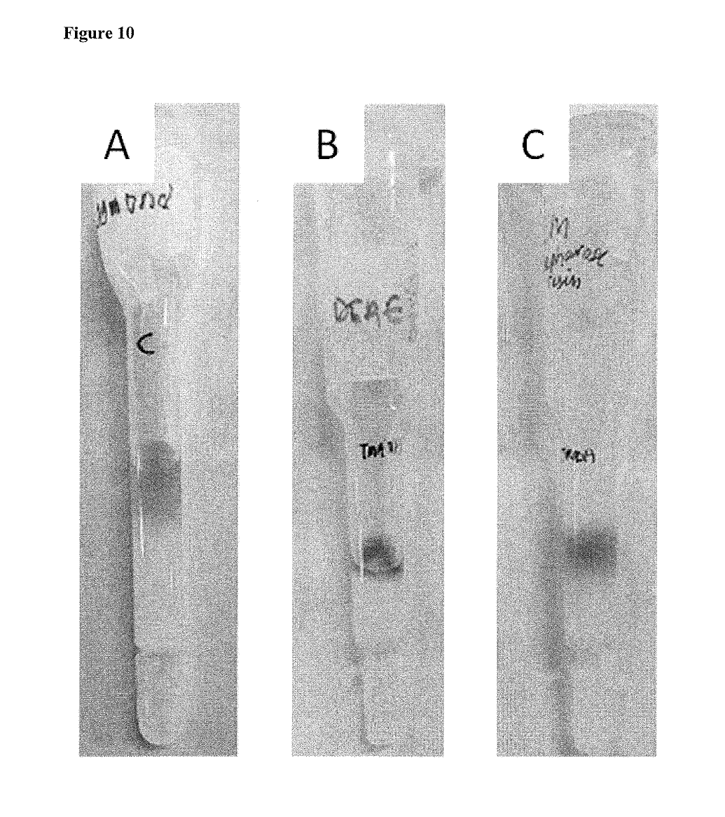

The non-specific binding affinity of the TMAOH coated iron oxide nanoparticles was tested with uncharged (G25), positively charged (DEAE) and negatively charged (CM) agarose-based resin columns (FIG. 10). The TMAOH coated nanoparticles showed non-specific binding to the G25 and DEAE resins but was not bound to the CM resin. This could be explained by charge repulsion between negatively charged TMAOH coating and the CM resin.

Protocol for Ligand Exchange of TMAOH Coated Iron Oxide Nanoparticles with Peptide Ligands

The protection of the iron oxide nanoparticles with a dense peptide self-assembled monolayers is not only dependent on the choice of peptide ligand, but highly affected by the experimental conditions. Hence the ligand exchange of the TMAOH coated iron oxide nanoparticles with all the peptide ligands from the library was performed using five protocols varying from the presence of electrolytes (NaCl), buffer (PBS, HEPES) or detergent (Tween 20). The protocols are presented below.

### H₂O as Solvent Only

The required ligand (5 mM, 40 μl) was dissolved in 165 μl of H₂O before the addition of the TMAOH coated iron oxide nanoparticles (1 mM iron content, 900 μl). The suspension was mixed overnight on a programed stirrer prior to concentration by centrifugation and purification on a G25 resin. The yield of the reaction was calculated by comparing the absorption maximal of the purified iron oxide nanoparticle solution with the initial solution.

### H₂O/tween 20 Solution

The required ligand (5 mM, 40 μl) was dissolved in 160 μl of H₂O and 5 μl of 1% tween 20 before the addition of the TMAOH coated iron oxide nanoparticles (1 mM iron content, 900 μl). The suspension was mixed overnight on a programed stirrer prior to concentration by centrifugation and purification on a G25 resin. The yield of the reaction was calculated by comparing the absorption maximal of the purified iron oxide nanoparticle solution with the initial solution.

### NaCl/tween 20 Solution

The required ligand (5 mM, 40 μl) was dissolved in 60 μl of H₂O, 100 μl of 1 M NaCl and 5 μl of 1% Tween 20, before the addition of the TMAOH coated iron oxide nanoparticles (1 mM iron content, 900 μl). The suspension was mixed overnight on a programed stirrer prior to concentration by centrifugation and purification on a G25 resin. The yield of the reaction was calculated by comparing the absorption maximal of the purified iron oxide nanoparticle solution with the initial solution.

### HEPES/tween 20 Solution

The required ligand (5 mM, 40 μl) was dissolved in 60 μl of H₂O, 100 μl of HEPES (10×) and 5 μ1 of 1% Tween 20, before the addition of the TMAOH coated iron oxide nanoparticles (1 mM iron content, 900 μl). The suspension was mixed overnight on a programed stirrer prior to concentration by centrifugation and purification on a G25 resin. The yield of the reaction was calculated by comparing the absorption maximal of the purified iron oxide nanoparticle solution with the initial solution.

### PBS/tween 20 Solution

The required ligand (5 mM, 40 μl) was dissolved in 60μl of H₂O, 100 μl of PBS (10×) and 5 μl of 1% Tween 20, before the addition of the TMAOH coated iron oxide nanoparticles (1 mM iron content, 900 μl). The suspension was mixed overnight on a programed stirrer prior to concentration by centrifugation and purification on a G25 resin. The yield of the reaction was calculated by comparing the absorption maximal of the purified iron oxide nanoparticle solution with the initial solution.

### Evaluation of Single Peptide Ligand Shells

The results of the evaluation of single peptide ligand from the library to efficiently coat and stabilize iron oxide nanoparticles is presented. Due to the large volume of data obtained from the screening, only a summary of the most important results and learning points is shown.

### Screening of Single Peptide Ligand Shells Prepared in Water Only

The evaluation of all the entire peptide ligand library was first performed with a protocol that does not contain and surfactant or electrolyte in order to simplify the screening and focus on the potential of the peptide ligand tested to ligand exchange with the TMAOH coating. A summary of the results obtained is presented in the Table 3. In this table, the yields and the normalized aggregation parameter of each preparation of peptide ligand coated iron oxide nanoparticles is presented and helped determined the most effective peptide ligands for ligand exchanging with the TMAOH coating. It is important to note that TMAOH coated iron oxide nanoparticles do not protect from non-specific binding to the agarose-based resin G25 and bind strongly to the column.

TABLE 3
Yield and NAP of ligand coated iron oxide
nanoparticles prepared using water only.

| Ligand | Yield (%) (NAP) | |
|---|---|---|
| L1 | 32 | (1.25 ± 0.08) |
| L2 | 44 | (1.31 ± 0.04) |
| L3 | 0 | |
| S1 | 2 | |
| S3 | 0 | |
| S5 | 49 | (1.21 ± 0.21) |
| S6 | 0 | |
| S7 | 80 | (1 ± 0.02) |
| S8 | 91 | (1 ± 0.01) |
| S9 | 8 | |
| S11 | 0 | |
| S13 | 80 | (1.23 ± 0.13) |
| S14 | 88 | (1.07 ± 0.008) |

Hence, this first screening allowed for the identification of 4 successful peptide ligands that enabled for the preparation of single peptide ligand shell on iron oxide nanoparticles with a high yield (above 80%) and a low normalized aggregation parameter (˜1). The list of peptide ligands is presented here:

peptide S7, PO₃H₂—O—CH₂—CO-Ser(PO₃H₂)—NH-PEG₄-OH

peptide S8, PO₃H₂—O—CH₂—CO-Ser(PO₃H₂)—NH-PEG₄-ol

peptide S13, H-Ser(PO₃H₂)-Val-Val-Val-Thr-ol

peptide S14, PO₃H₂—O—CH₂—CO-Ser(PO₃H₂)-Val-Val-Val-Thr-ol

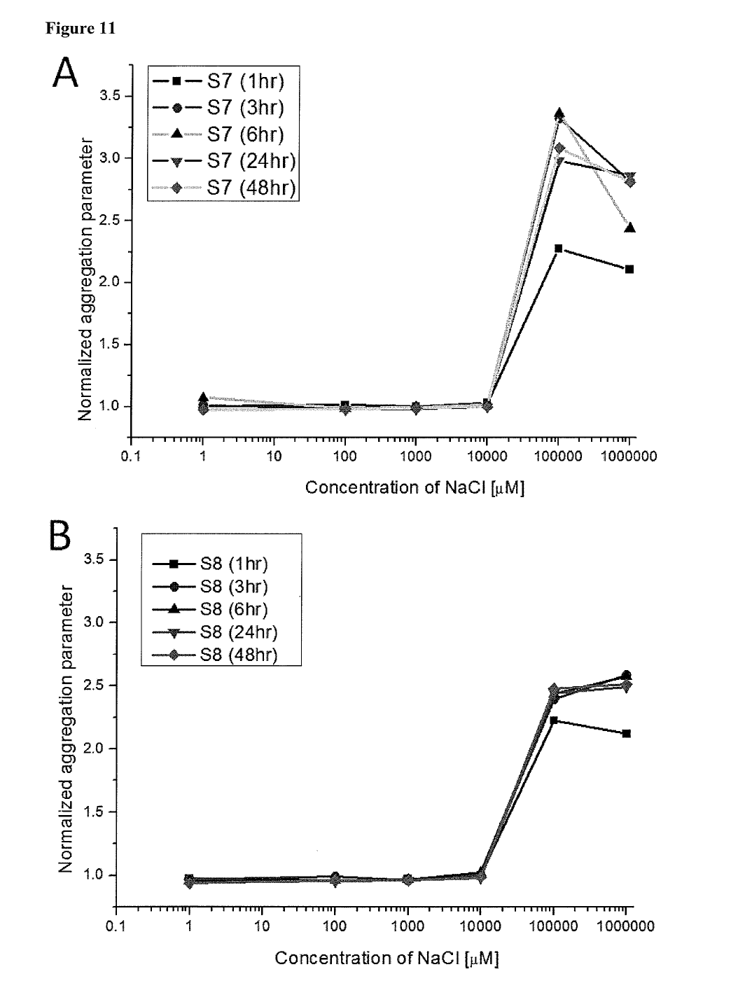

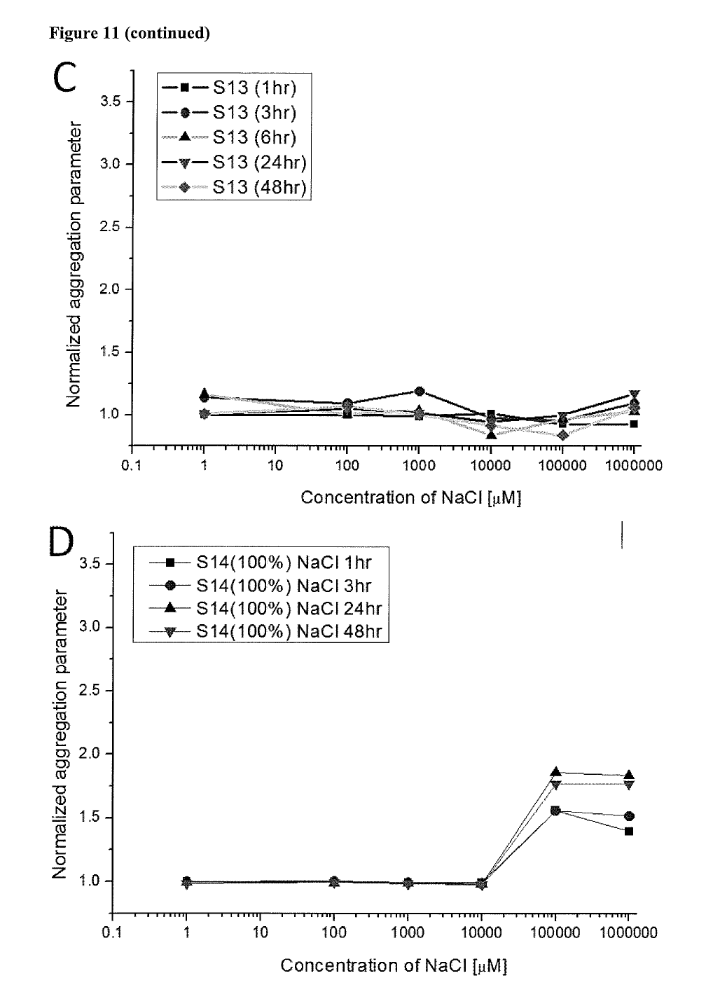

The four single peptide ligand shells were evaluated for their potential to stabilize the iron oxide nanoparticles against electrolyte-induced aggregation. The nanoparticles were mixing with a range of concentration of NaCl (0.1 mM to 1 M) at room temperature. The stability of the nanoparticles was followed by UV-visible spectrophotometry and the normalized aggregation parameters were determined. The results presented in FIG. 11 showed that, although the single peptide shells protected the nanoparticles efficiently enough to allow for their purification by size-exclusion chromatography with high yield, none of them presented sufficient stability in concentration of NaCl higher than 10 mM. The aggregation of all nanoparticles were observed also in PBS buffer (with 100 mM NaCl) after few hours.

Other protocols using different buffers and surfactants were tested then in order to improve the preparation of the single peptide coated nanoparticles and allow for a higher stability against electrolyte-induced aggregation.

Increase of Packing Density of single Peptide Ligand Shells Prepared in PBS and Tween 20

The most promising protocol to prepare single peptide ligand coating on iron oxide nanoparticles used PBS buffer (with 150 mM NaCl) and the surfactant Tween 20. The results shown in Table 4 demonstrated again that the double phosphorylated peptide ligand S14 provided a high yield of preparation of peptide coated nanoparticles after purification by size-exclusion chromatography.

TABLE 4
Yield and NAP of ligand coated iron oxide
nanoparticles prepared using PBS and Tween 20.

| Ligand | Yield (%) (NAP) |
|---|---|
| S7 | 63 (1.33 ± 0.08) |
| S13 | 17 (2.23 ± 0.34) |
| S14 | 94 (1.24 ± 0.02) |

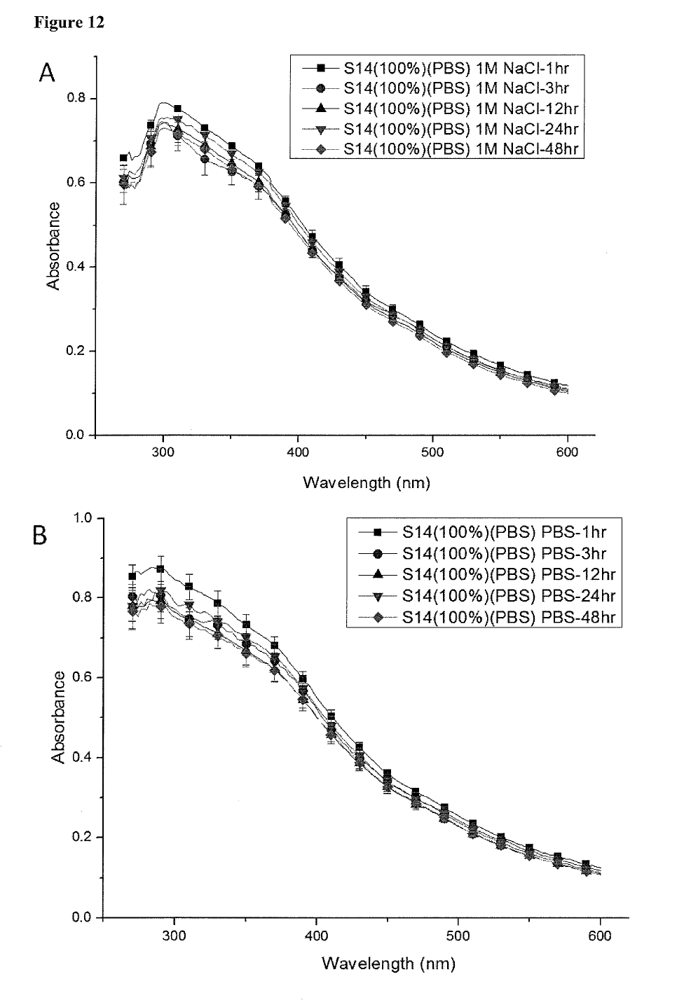

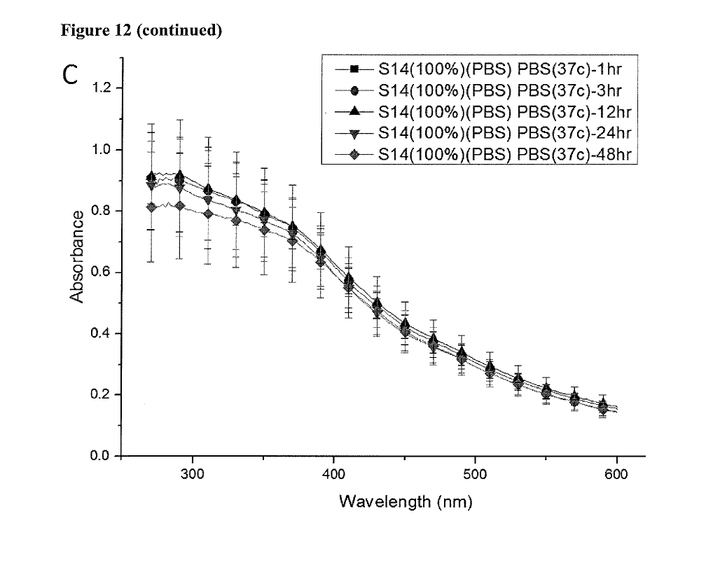

The single peptide shell made of the peptide S14 was tested for its ability to protect the iron oxide nanoparticles from electrolyte-induced aggregation. The results presented in FIG. 12 show that the peptide S14 enabled high stability of the nanoparticles at room temperature in high concentration of NaCl (1 M) and PBS buffer over two days. Most importantly, the same high stability was observed in PBS buffer at 37° C. for two days. This represents a promising result since the utilization of the peptide coated iron nanoparticles in vitro and in vivo are performed at 37° C.

### Evaluation of Mixed Peptide Ligand Shells

Based on the experience in the preparation of stable peptide coated gold nanoparticles, it has been previously demonstrated that mixtures of peptides and thin alkane ethylene glycol ligands may greatly improve the stability and reduce the non-specific binding property of nanoparticles. The library contains three thin phosphorylated alkane ethylene glycol ligands (L1, L2, L3). All the possible combinations with the peptides S9, S11, S13 and S14 mixed with the individual ligands L1, L2 and L3, were tested with the molar ratios peptide:ligand of 70:30, 50:50 and 30:70.

The results of the evaluation of single peptide ligand from the library to efficiently coat and stabilize iron oxide nanoparticles is presented. Due to the large volume of data obtained from the screening, only a summary of the most important results and learning points is shown.

### Screening of Mixed Peptide Ligand Shells Prepared in Water Only

Similarly to the screening of single ligand shells, the evaluation of mixed peptide ligand shells was first performed with a protocol that does not contain and surfactant or electrolyte in order to simplify the screening and focus on the potential of the peptide ligand mixtures tested to ligand exchange with the TMAOH coating and stabilize iron oxide nanoparticles. A summary of the results obtained is presented in the Table 5. In this table, the yields and the normalized aggregation parameter of each preparation of peptide ligand coated iron oxide nanoparticles is presented.

TABLE 5
Yield and NAP of mixed peptide ligand coated iron
oxide nanoparticles prepared using water only.

| | | Molar ratio | | |
|---|---|---|---|---|
| Peptide | Ligand | Peptide:Ligand | Yield (%) (NAP) | |
| Peptide S9 | Ligand L1 | 70:30 | 75.3 | (1.18 ± 0.2) |
| | | 50:50 | 50 | (1 ± 0.22) |
| | | 30:70 | 79 | (1.33 ± 0.03) |
| | Ligand L2 | 70:30 | 26 | (1.12 ± 1) |
| | | 50:50 | 69 | (1.14 ± 0.2) |
| | | 30:70 | 55 | (1 ± 0.16) |
| | Ligand L3 | 70:30 | 19 | (NA) |
| | | 50:50 | 100 | (1 ± 0.1) |
| | | 30:70 | 13 | (NA) |
| Peptide S11 | Ligand L1 | 70:30 | 0 | |
| | | 50:50 | 0 | |
| | | 30:70 | 42.9 | (0.83 ± 0.1) |
| | Ligand L2 | 70:30 | 0 | |
| | | 50:50 | 0 | |
| | | 30:70 | 0 | |
| | Ligand L3 | 70:30 | 0 | |
| | | 50:50 | 33 | (0.64 ± 0.05) |
| | | 30:70 | 42.9 | (0.83 ± 0.1) |
| Peptide S13 | Ligand L1 | 70:30 | 5 | (2.9 ± 2.6) |
| | | 50:50 | 0 | |
| | | 30:70 | 49 | (1 ± 0.3) |
| | Ligand L2 | 70:30 | 27 | (3.7 ± 3) |
| | | 50:50 | 4 | (4.1 ± 4) |
| | | 30:70 | 36 | (0.7 ± 1) |
| | Ligand L3 | 70:30 | 73 | (0.83 ± 0.025) |
| | | 50:50 | 91 | (0.84 ± 0.08) |
| | | 30:70 | 81 | (0.81 ± 0.05) |
| Peptide S14 | Ligand L1 | 70:30 | 99 | (1.17 ± 0.07) |
| | | 50:50 | 90 | (1.5 ± 0.5) |
| | | 30:70 | 95 | (1.6 ± 0.35) |
| | Ligand L2 | 70:30 | 100 | (1.4 ± 0.23) |
| | | 50:50 | 86 | (1.1 ± 0.13) |
| | | 30:70 | 88 | (1.2 ± 0.03) |
| | Ligand L3 | 70:30 | 100 | (1.1 ± 0.1) |
| | | 50:50 | 72 | (1.59 ± 0.01) |
| | | 30:70 | 83 | (1.61 ± 0.03) |

In this first screening, it is clear that the most effective mixtures for ligand exchange of TMAOH coating are made of the peptide S14. Most of the combinations with the ligands L1, L2 or L3, provided a high yield (above 80%) and a fairly low normalized aggregation parameter (1 to 1.5). Moreover, highest content of peptide S14 in the mixtures gave the highest yields.

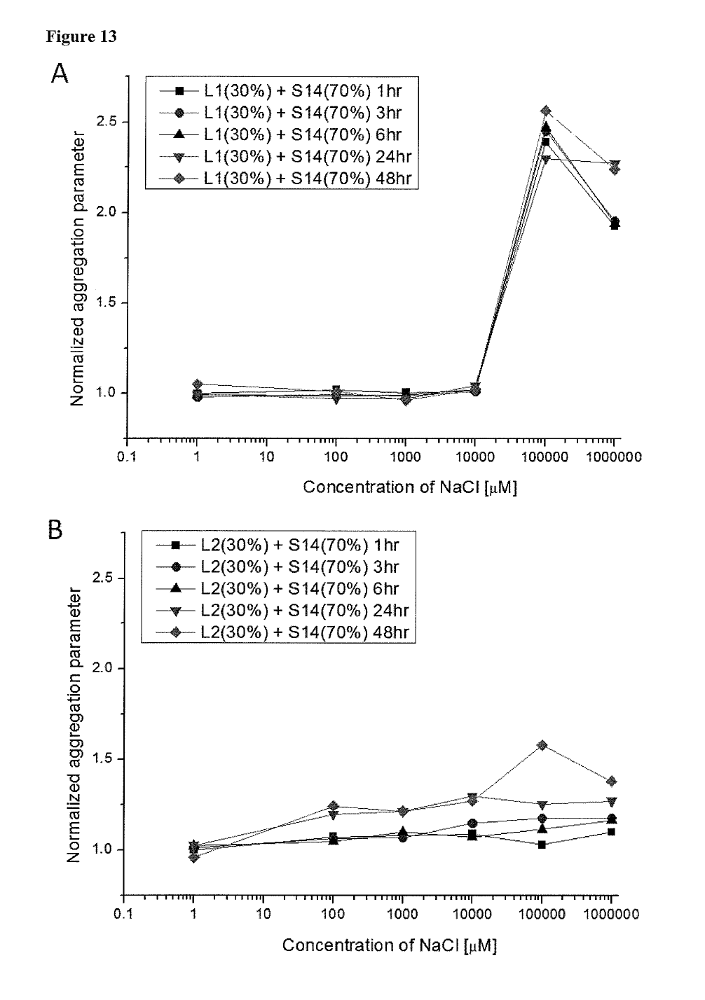

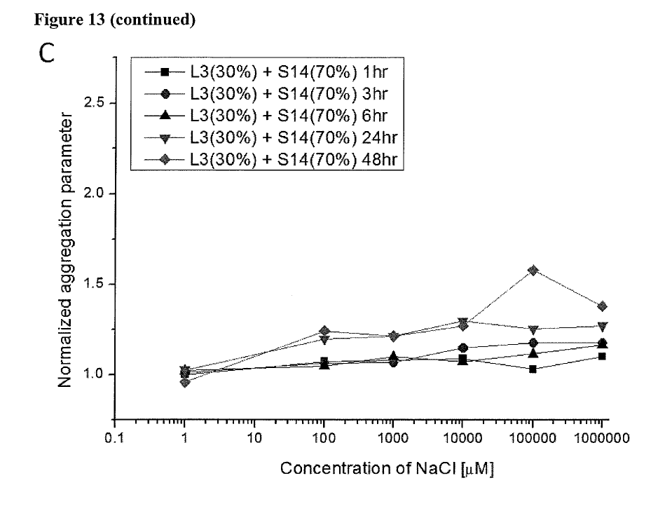

All the mixed peptide ligand shells providing sufficient yield of nanoparticles were evaluated for electrolyte-induced aggregation stability by mixing the nanoparticles with a range of concentration of NaCl (0.1 mM to 1 M) at room temperature. The stability of the nanoparticles was followed by UV-visible spectrophotometry and the normalized aggregation parameters were determined Here, only the results obtained with the peptide S14 mixed shells with a molar ratio of peptide:ligand of 70:30 are presented in FIG. 13. The results showed clearly that the most stable mixture is made of the peptide S14 in combination with the ligand L1.

Most interestingly, the chemical structures of the peptide and ligand providing the best results so far and used in combination, i.e., peptide S14 (PO₃H₂—O—CH₂-CO-Ser(PO₃H₂)-Val-Val-Val-Thr-ol) and ligand L1 ((HO)₂—PO—S—C₁₁-EG₃-OH), are very similar to the currently most stable mixture of ligand used to prepare biocompatible gold nanoparticles, i.e., H-Cys-Val-Val-Val-Thr-ol and HS—C₁₁-EG₄-OH.

However, similar to the preparation of single peptide ligand shells, although the mixed peptide shells protected the nanoparticles efficiently enough to allow for their purification by size-exclusion chromatography with high yield, none of them presented sufficient stability in concentration of NaCl higher than 10 mM at this stage. The aggregation of all nanoparticles were observed also in PBS buffer (with 100 mM NaCl) after few hours.

Other protocols using different buffers and surfactants were tested then in order to improve the preparation of the single peptide coated nanoparticles and allow for a higher stability against electrolyte-induced aggregation.

### Increase of Packing Density of Mixed Peptide Ligand Shells Prepared in PBS and Tween 20

The most promising protocol to prepare mixed peptide ligand coating on iron oxide nanoparticles used PBS buffer (with 150 mM NaCl) and the surfactant Tween 20. The results shown in Table 6 demonstrated again that the double phosphorylated peptide S14 mixed with the ligand L1 provided a higher yield of preparation of peptide coated nanoparticles after purification by size-exclusion chromatography.

TABLE 6
Yield and NAP of mixed peptide ligand coated iron oxide
nanoparticles prepared using PBS and Tween 20.

| Peptide ligand | |
|---|---|
| mixture | Yield (%) (NAP) |
| S14(70%) + L1(30%) | 75 (1.24 ± 0.01) |
| S14(70%) + L3(30%) | 52 (1.67 ± 0.06) |

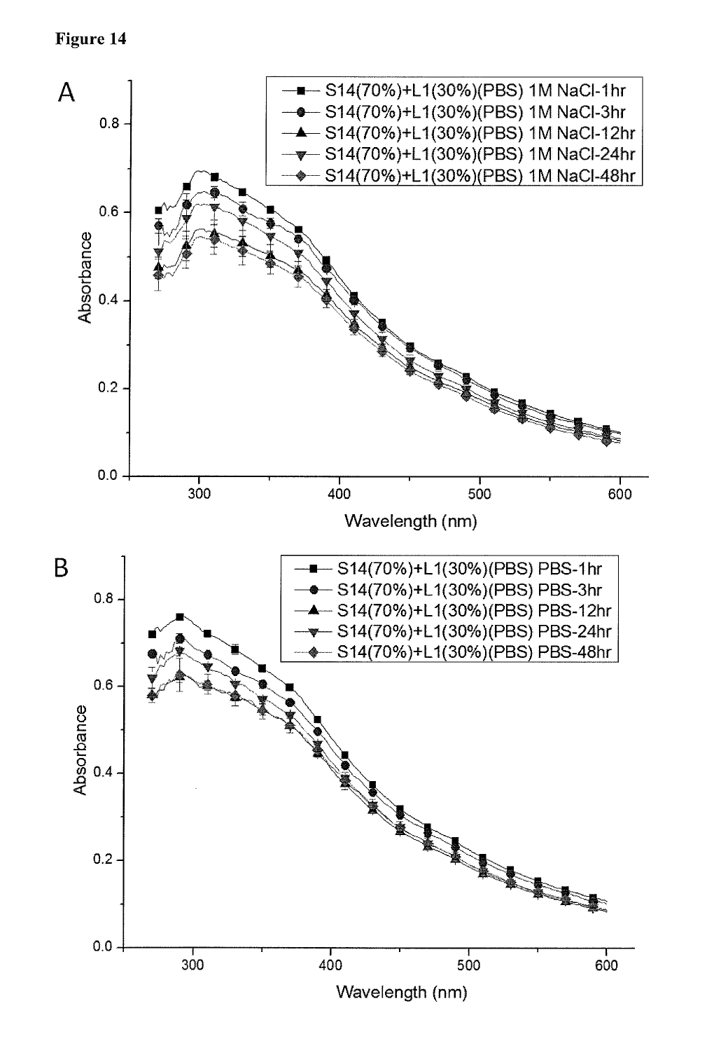

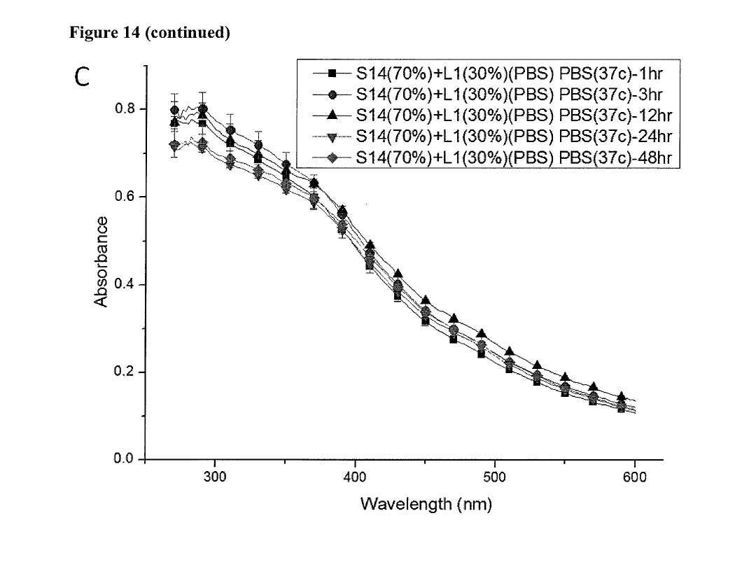

The mixed peptide ligand shell made of the peptide S14 and ligand L1 was tested for its ability to protect the iron oxide nanoparticles from electrolyte-induced aggregation. The results presented in FIG. 14 show that this mixed peptide shell enabled high stability of the nanoparticles at room temperature in high concentration of NaCl (1 M) and PBS buffer over two days. Most importantly, the same high stability was observed in PBS buffer at 37° C. for two days. This represents again a promising results since the utilisation of the peptide coated iron nanoparticles in vitro and in vivo are performed at 37° C.

### Conclusions and Perspectives

In conclusion, a total of 16 single and 36 mixed peptide ligand shells were evaluated for the preparation of stable peptide coated iron oxide nanoparticles. Currently, the most promising results were obtained with the double phosphorylated peptide S14, PO₃H₂—O—CH₂—CO-Ser(PO₃H₂)-Val-Val-Val-Thr-ol, as single ligand shell or mixed with the ligand L1, (HO)₂—PO—S—C₁₁-EG₃-OH. In both case, the nanoparticles were most stable using a protocol with PBS buffer and the surfactant Tween 20. The peptide coated nanoparticles showed good stability against electrolyte-induced aggregation in high concentration of NaCl (1 M) and in PBS buffer over two day. Most importantly, the same stability was also observed in PBS buffer at physiological temperature (37° C.) over two days which is a prerequisite for in vitro and in vivo experiments.

The most promising peptide coated iron oxide nanoparticles are now evaluated for their non-specific binding property against charged agarose-based resins. In vitro experiments will be also done to validate their non-specific binding to cells and their cytotoxicity.

Moreover, additional peptides have been designed and synthesized to further improve the preparation of biocompatible nanoparticles. The complete evaluation is now achieved.

### Imaging Phantoms for Evaluation of Magnetic Properties of Coated SPIONs.

Imaging phantoms experiments have been done to evaluate the magnetic properties of peptide coated iron oxide nanoparticles. The initial phantom measurements were performed with the most stable peptide coated nanoparticles prepared with a protocol using water only as solvent.

### Phantom Preparation

Samples are made into 6 concentrations each by serial dilution. Briefly, 550-600 uL of the stock sample is transferred into a 1.5 mL Eppendorf and an equal amount of ddH₂O is added. The diluted sample is further serial diluted into 5 more concentration to make a total of 6 different concentrations.

### Magnetic Resonance Relaxivity Measurements

Magnetic resonance relaxivity of the nanoparticles were evaluated by using a 7-Tesla Bruker Clinscan MRI system. T2 relaxation times were determined from a multiecho spin-echo sequence (repetition time (TR): 4000 ms; TE: 17.9-250.6 ms). (r2) relaxivities were obtained from the slope of 1/T2 versus molar [Fe] concentration plots.

### Results

TABLE 7
Summary of R2 values obtained.

| Coating | R2 value (mM⁻¹s⁻¹) | Comments |
|---|---|---|
| TMAOH | 104.8 | R² = 0.995 5/6 data points |
| Resovist | 212.0 | R² = 0.987 6 data points |
| S7 | 139.0 | 4/6 data points |
| S8 | 253.4 | 4/6 data points |
| S14 | 266.9 | 4/6 data points |
| L1(30%) + S9(70%) | NIL | Precipitated |
| L3(70%) + S13(30%) | 267.2 | 4/6 data points |
| L3(50%) + S13(50%) | NIL | Precipitated |
| L3(30%) + S13(70%) | NIL | Precipitated |
| L1(70%) + S14(30%) | 84.22 | 4/6 data points |
| L1(50%) + S14(50%) | 194.0 | 4/6 data points |
| L1(30%) + S14(70%) | 145.8 | 4/6 data points |
| L2(70%) + S14(30%) | 202.4 | 3/6 data points |
| L2(50%) + S14(50%) | 202.3 | 4/6 data points |
| L2(30%) + S14(70%) | NIL | Precipitated |
| L3(30%) + S14(70%) | NIL | Precipitated |

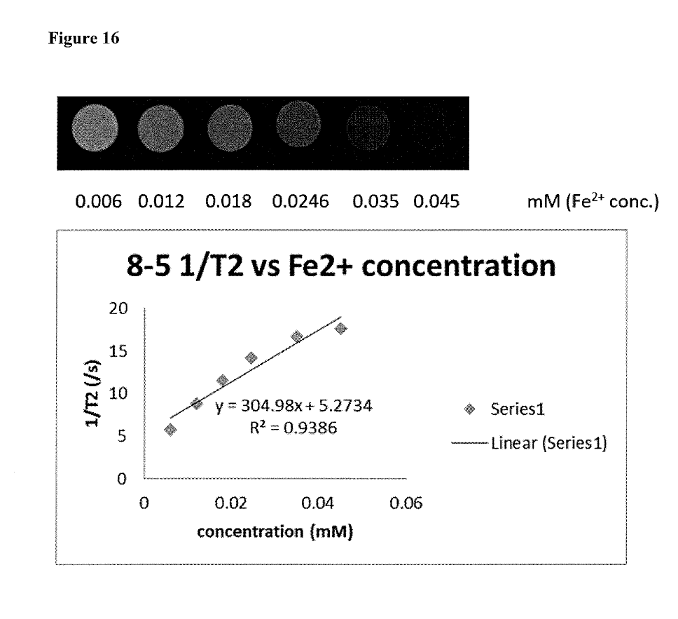

For the samples without R2 values, MRI measurements of those samples showed no linear trend when 1/T₂ is plotted against the Fe²⁺ concentration (FIG. 16). The samples in these phantoms precipitated in the syringe.

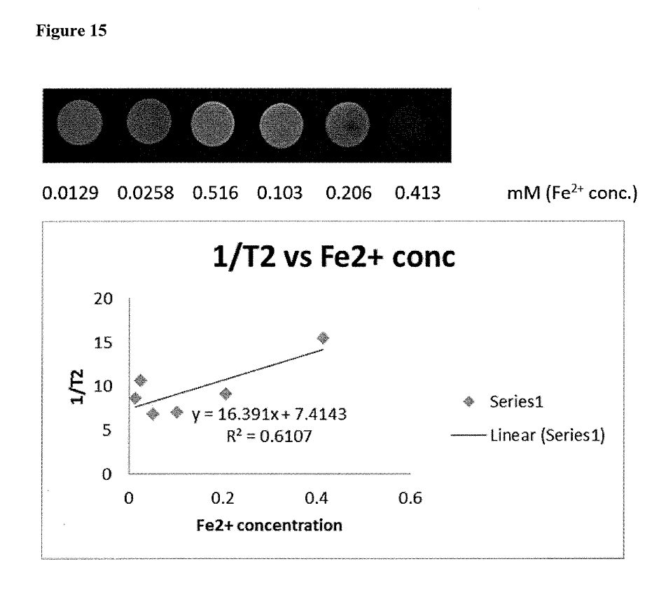

The image of FIG. 15 shows the MRI intensities of the L1(30%)+S9(70%) mixed peptide coated nanoparticles phantoms in different concentrations (without trend).

The image of FIG. 16 shows the MRI intensities of the peptide S7 coated iron oxide nanoparticles phantoms in different concentrations (with trend).

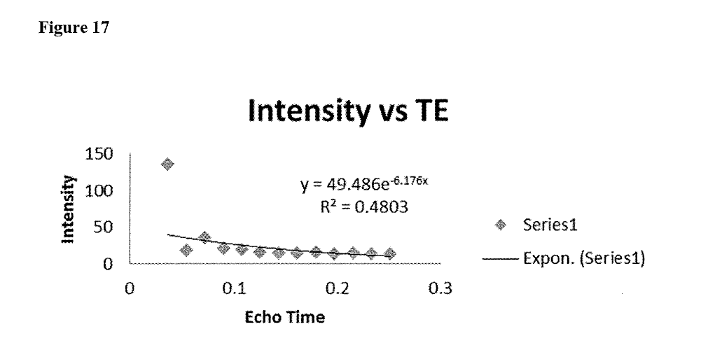

For the rest of the samples, the 1^{st} 2-3 concentrations had bad fittings on the decay curve (FIG. 17), resulting in the loss of 2-3 data points.

In order to generate more data points, the samples were further serial diluted to create 3 more phantoms of lower concentrations. Results obtained were not satisfactory. The lower concentrations were too low, with contrast similar to the water control.

3 samples together with the starting material (TMAOH coated iron oxide nanoparticles) was selected from the list of samples based on the initial R2 results as well as their observed stability. Sample was recovered from the highest concentration phantom and used to prepare 6 different concentrations in a tighter range (ie. No serial dilution).

The phantoms were sent for MRI measurements on the same day they were prepared. Parameters used were the same as previous MRI phantom measurements.

| Coating | R2 value (mM⁻¹s⁻¹) | Comments |
|---|---|---|
| S7 | 304.9 | R² = 0.938 |
| S8 | 269.0 | R² = 0.998 |
| L1(30%) + S14(70%) | 85.28 | R² = 0.906 |
| TMAOH | 104.8 | R² = 0.995 5/6 data points |

### Conclusions and Perspectives

The initial data obtained that the most stable nanoparticles allowed for the determination of their relaxivity. The R2 values of the peptide coated iron oxide nanoparticles in those experiments were relatively high (above 200 mM⁻¹.s⁻¹) in most case which is comparable to the Resovist iron oxide nanoparticles, a FDA approved MRI contrast agent.

The preparation of more stable nanoparticles is now completed allow for a better reading of their performance in MRI phantom experiments.

### In Vitro Cytotoxicity Assay of Coated SPIONs.

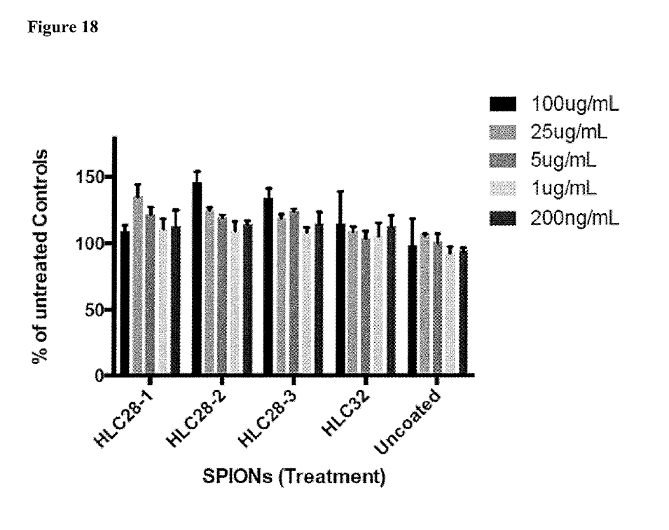

A series of most stable peptide coated iron oxide nanoparticles were evaluated in vitro. The MTT assay (FIG. 18) with BT474 breast cancer cells showed that the nanoparticles did not present any cytotoxicity at high concentrations (tested up to 100 μg/mL of iron).

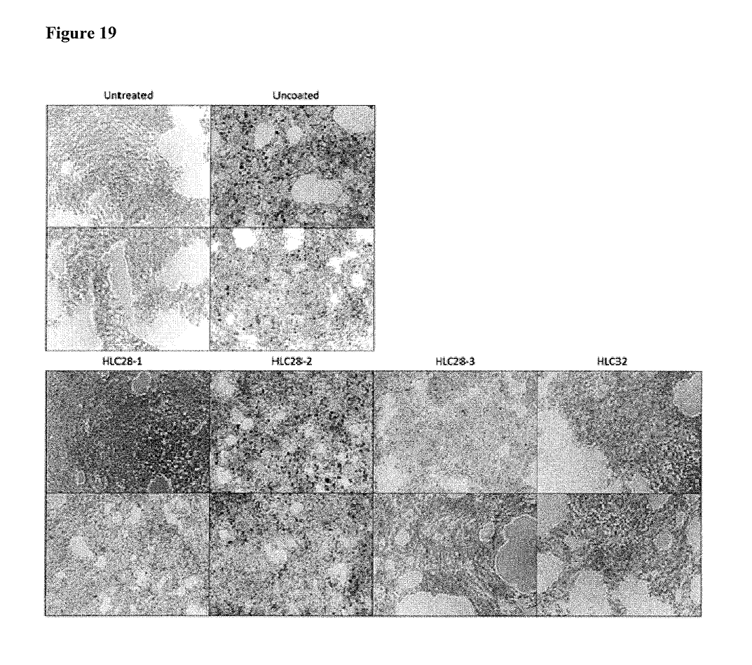

The non-specific staining has also been conducted with BT474 breast cancer cells with the best performing peptide coated iron oxide nanoparticles. The staining of the cells with Prussian blue the assay helped identified the most performing peptide coatings (FIG. 19).The invention has been described broadly and generically herein. Each of the narrower species and subgeneric groupings falling within the generic disclosure also form part of the invention. This includes the generic description of the invention with a proviso or negative limitation removing any subject-matter from the genus, regardless of whether or not the excised material is specifically recited herein. Other embodiments are within the following claims. In addition, where features or aspects of the invention are described in terms of Markush groups, those skilled in the art will recognize that the invention is also thereby described in terms of any individual member or subgroup of members of the Markush group.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of preferred embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. The word “comprise” or variations such as “comprises” or “comprising” will accordingly be understood to imply the inclusion of a stated integer or groups of integers but not the exclusion of any other integer or group of integers. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The content of all documents and patent documents cited herein is incorporated by reference in their entirety.

1. A ligand compound having the structure A-B-C, wherein
(a) A represents a mono- or polyphosphorylated amino acid linked to part B by its amino group to form an amide bond;
B represents
(i) a carboxylic acid linked to part A by its acidic group to form the amide bond, and
(ii) an amino acid or peptidyl group of 2 to 10 amino acids, an alkyl or alkenyl group comprising 1 to 26 carbon atoms, a polyethylene glycol group comprising 1 to 26 carbon atoms or a combination thereof covalently linked to the carboxylic acid; and
C represents a hydrophilic group covalently linked to the group of B (ii); or
(b) A represents a mono- or polyphosphorylated amino acid linked to part B by its carboxylic acid to form an amide bond;
B represents an amino acid or peptidyl group of 2 to 10 amino acids, an amino substituted alkyl or alkenyl group comprising 1 to 26 carbon atoms, an amino substituted polyethylene glycol group comprising 1 to 26 carbon atoms or a combination thereof covalently linked to A by their amino group;
C represents a hydrophilic group covalently linked to the group of B.

2. The ligand compound according to claim 1, wherein
(a) the phosphorylated amino acid is phosphoserine, phosphothreonine or phosphotyrosine; and/or
(b) the phosphorylated amino acid of the ligand compound according to claim 1(b) is an amino acid comprising attached to its N-terminus the moiety PO₃H₂—O—CH₂—CO—.

3. The ligand compound according to claim 1 or 2, wherein the carboxylic acid is an amino acid.

4. The ligand compound according to any one of claims 1-3, wherein the hydrophilic group is a group comprising a carboxyl group, a hydroxyl group or an amine group.

5. The ligand compound according to any one of claims 1-4, wherein the hydrophilic group is an amino acid derivative selected from the group consisting of aspartyl, glutaminyl, arginyl, histidyl and lysyl.

6. The ligand compound according to any one of claims 1-5, wherein the ligand compound is functionalized by the attachment of an additional group.

7. The ligand compound according to claim 6, wherein the additional group is selected from the group consisting of a dye, a radionuclide, a pharmaceutical agent, a biotherapeutic agent, a chemotherapeutic agent, a radiotherapeutic agent, and combinations thereof.

8. The ligand compound according to claim 1, 2 or 4, wherein
(a) the compound having the structure of formula (I) 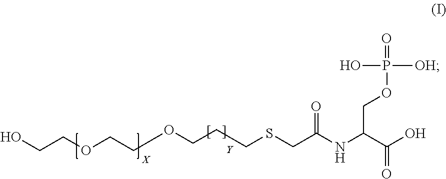
wherein X and Y are independently from each other an integer ranging from 1-10; or
(b) the ligand compound is selected from the group consisting of H-Ser-(PO₃H₂)—NH-PEG₄-ol, PO₃H₂—O—CH₂—CO-Gly-NH-PEG₄-OH, PO₃H₂—O—CH₂—CO-Ser(PO₃H₂)—NH-PEG₄-OH, PO₃H₂—O—CH₂—CO-Ser(PO₃H₂)—NH-PEG₄-ol, H-Ser(PO₃H₂)-Ser-Ser-Ser-Ser-ol, H-Ser-(PO₃H₂)-Val-Val-Val-Thr-ol and PO₃H₂—O—CH₂—CO-Ser(PO₃H₂)-Val-Val-Val-Thr-ol.

9. The ligand compound according to claim 8(a), wherein X is 3 and Y is 9.

10. A coated metal nanoparticle comprising a core metal nanoparticle that is coated with a plurality of ligand compounds according to any one of claims 1-9.

11. The coated metal nanoparticle according to claim 10, wherein the plurality of ligand compounds according to any one of claims 1-9 comprises a mixture of at least two structurally different ligand compounds.

12. The coated metal nanoparticle according to claim 10 or 11, wherein the core metal nanoparticle is a metal oxide nanoparticle, preferably iron oxide nanoparticle and more preferably a superparamagnetic iron oxide nanoparticle (SPION).

13. Use of a ligand compound according to any one of claims 1-9 for coating a metal nanoparticle.

14. Method of producing a coated metal nanoparticle according to any one of claims 10-12 comprising:
(a) providing a core metal nanoparticle and a plurality of ligand compounds according to any one of claims 1-9; and
(b) combining the core metal nanoparticle and the plurality of ligand compounds under conditions that allow the formation of the coated metal nanoparticle according to any one of claims 10-12.

15. The method according to claim 14, wherein said method comprises prior to step (a) encapsulation of the metal nanoparticle with an intermediate hydrophilic ligand, preferably tetramethylammonium hydroxide (TMAOH).

16. Coated metal nanoparticle according to any one of claims 10-12 for use as a medicament.

17. The coated metal nanoparticle according to claim 16, wherein the ligand compound is functionalized by the attachment of an additional group.

18. The coated metal nanoparticle according to claim 17, wherein the additional group is selected from the group consisting of a dye, a radionuclide, a pharmaceutical agent, a biotherapeutic agent, a chemotherapeutic agent, a radiotherapeutic agent, and combinations thereof.

19. Method of producing the ligand compound according to claim 8(a) comprising:
a) reacting a compound of formula (II) 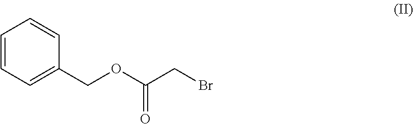 [SMILES: O=C(CBr)OCc1ccccc1]
with NaI to form a compound of formula (III) 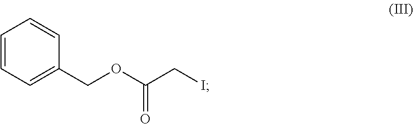 [SMILES: O=C(CI)OCc1ccccc1]
b) reacting the compound of formula (III) with a compound of formula (IV) 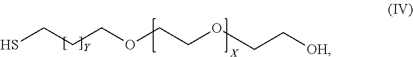
wherein X and Y are independently from each other an integer ranging from 1-10, to form a compound of formula (V) 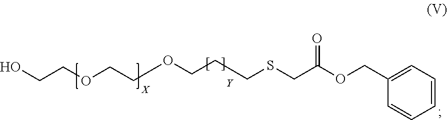
c) reacting the compound of formula (V) with Boc₂O to form a compound of formula (VI) 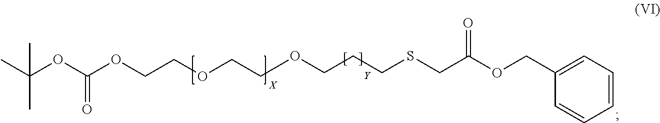
d) reacting the compound of formula (VI) with H₂ to form the a compound of formula (VII) 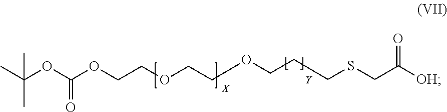
e) reacting the compound of formula (VII) with a compound of formula (VIII) 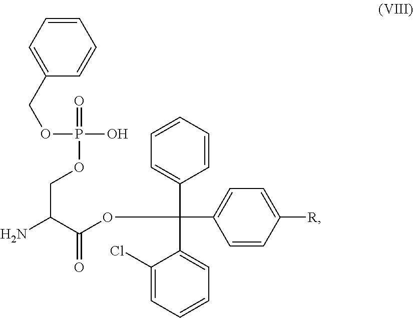
wherein R represents a resin,
to form a compound of formula (IX) 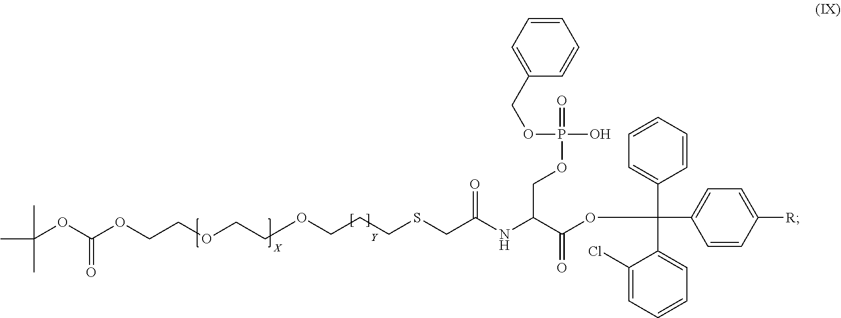
f) reacting the compound of formula (IX) with dichloromethane (DCM): trifluoroacetic acid (TFA) to form a compound of formula (X) 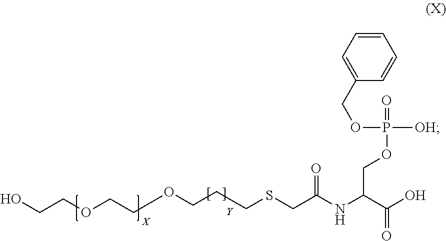
and
g) reacting the compound of formula (X) with H₂ to form the ligand compound according to claim 8(a).
